(19) United States
(12) Patent Application Publication
(10) Pub. No.: US 2023/0355765 A1
(43) Pub. Date: Nov. 9, 2023

(54) CHIMERIC ANTIGEN RECEPTOR DENDRITIC CELLS (CAR-DCS) AND METHODS OF MAKING AND USING SAME

(71) Applicant: Washington University, St Louis, MO (US)

(72) Inventor: CARL DESELM, St. Louis, MO (US)

(21) Appl. No.: 17/786,255

(22) PCT Filed: Dec. 16, 2020
(86) PCT No.: PCT/US2020/065378

§ 371 Date: Jun. 16, 2022

Related U.S. Application Data
(60) Provisional application No. 62/948,612, filed on Dec. 16, 2019.

(51) Int. Cl. A61P 35/00; A61K 39/00; C07K 14/725; C07K 16/28; C07K 14/705; C12N 9/12; C12N 5/0784

(52) U.S. Cl. CPC A61K 39/464462; A61K 39/4615; A61K 39/4622; A61K 39/4631; A61P 35/00; C07K 14/7051; C07K 14/70517; C07K 16/2866; C12N 5/0639; C12N 9/12; C12Y 207/10001; A61K 2239/15; A61K 2239/21; A61K 2239/22; C07K 2317/24; C07K 2317/33; C07K 2317/622; C07K 2319/02; C07K 2319/03; C07K 2319/033; C12N 2501/26; C12N 2510/00

(57) ABSTRACT

Among the various aspects of the present disclosure is the provision of compositions and methods of making modified chimeric antigen receptor dendritic cells (CAR-DCs) and methods of use thereof. CAR-DCs can be used for the treatment of tumors and cancers, particularly solid tumors (as well as liquid tumors, blood cancer, and metastatic cancer).

### CROSS-REFERNCE TO RELATED APPLICATIONS

This application claims benefit from U.S. Provisional Application Serial No. 62/948,612 filed on Dec. 16, 2019, which is incorporated herein by reference in its entirety.

### GOVERNMENTAL RIGHTS

This invention was made with government support under OD026427 awarded by the National Institutes of Health. The government has certain rights in the invention.

### FIELD OF THE TECHNOLOGY

Disclosed herein are compositions and methods of creating an adaptive immune response in a subject. In particular, the disclosure relates to dendritic cells genetically modified to express one or more chimeric antigen receptors (CARs) and methods of using the same for the treatment of cancer.

### REFERENCE TO SEQUENCE LISTING

This application contains a Sequence Listing that has been submitted in ASCII format via EFS-Web and is hereby incorporated by reference in its entirety. The ASCII copy, created on Dec. 14, 2020, is named 675958_ST25, and is 7.64 KB bytes in size.

### BACKGROUND

Harnessing an organism’s immune system to fight a disease such as cancer is a powerful approach. Recent work has identified two key immune checkpoint proteins displayed on the surface of T cells. These cell-surface receptors bind certain ligands displayed on other cells such as antigen-presenting cells (APCs) and recognize them as self, which leads to the attenuation of T cell activity and puts a brake on the immune response. Researchers have shown, during decades of work, that preventing these receptors on T cells from binding their ligands on APCs or tumor cells lifts the block and triggers an attack on the tumor cells.

For full activation of a T cell, this brake on negative immune modulation is necessary, but not sufficient. Another surface receptor, the T cell receptor (TCR), also needs to recognize and bind a ligand specifically derived from tumor cells and displayed on APCs. Evidence now exists that metastatic cancers are sometimes curable if a patient possesses antitumor T cells. Checkpoint inhibitors, which “release the breaks” on antitumor T cells, induce a complete response (CR) in up to 10-15% of metastatic melanoma and several other types of cancer, some of which are durable.

Chimeric antigen receptor (CAR) therapy has achieved great clinical success against hematological malignancies. It is based on synthetic receptors with both antigen recognition and signal transduction functions. The single-chain variable fragment (scFv) in a CAR retains its antigen recognition specificity from the variable regions of the heavy and light chains of the original monoclonal antibody. Meanwhile, signal transduction of the CAR construct largely depends on the signaling domains of the original immune receptors. CAR T cells exhibit a remarkable 80-100% CR in end-stage relapsed acute lymphoblastic leukemia (ALL) patients, but a 1% CR in solid tumors.

Understanding and overcoming the failures of each therapy may help bring durable remissions to the remainder of cancer patients. Reasons for failure are multifactorial, but checkpoint inhibitors are ineffective at baseline if patients do not first have an adaptive antitumor T cell response, which is more likely in tumors with higher mutational load. Solid tumors escape CAR T recognition if not all cells express the target antigen. Successfully creating an adaptive immune response in patients would overcome the failures of both types of immunotherapy. However, achieving this remains elusive.

Therefore, a need in the art exists for compositions and methods for specifically targeting various cancer or tumor cells, while creating an adaptive immune response by harnessing antitumor T cells.

### SUMMARY

Among the various aspects of the present disclosure is the provision of a chimeric antigen receptor dendritic cell (CAR-DC) and methods of making and using CAR-DCs.

An aspect of the present disclosure provides for a modified cell comprising a chimeric antigen receptor (CAR), wherein the CAR comprises: an antigen binding domain; a transmembrane domain; an intracellular domain comprising a FMS-like tyrosine kinase 3 (Flt3) signaling domain; and/or the modified cell is a dendritic cell or a precursor or a progenitor cell thereof.

Another aspect of the present disclosure provides for chimeric antigen receptor (CAR) construct comprising: (i) an antigen-binding domain; (ii) a transmembrane domain; and/or (iii) an intracellular signaling domain comprising an FMS-like tyrosine kinase 3 (Flt3) signaling domain, wherein the CAR construct is capable of being expressed or functioning in a dendritic cell (DC) or a precursor or a progenitor cell thereof.

In some embodiments, the dendritic cell is selected from a cDC1 cell or precursor or progenitor thereof.

Another aspect of the present disclosure provides for a modified cell comprising a first nucleic acid sequence encoding a CAR or a second nucleic acid sequence encoding the antigen binding domain, the transmembrane domain, and the intracellular domain.

In some embodiments, the first intracellular nucleic acid sequence encodes a protein product comprising Flt3 or a Flt3-based protein product or subsequent intracellular nucleic acid sequence encodes a protein product comprising Flt3 or a Flt3-based protein product.

In some embodiments, the CAR further comprises a signal peptide or an additional extracellular domain.

In some embodiments, the modified cell is a conventional type 1 dendritic cell (cDC1).

In some embodiments, the modified cell is capable of antigen cross-presentation, an adaptive antitumor immune response, or activation of antitumor T cells.

In some embodiments, the antigen binding domain comprises an antibody or fragment thereof.

In some embodiments, the antibody has a binding affinity to a tumor cell antigen.

In some embodiments, the tumor cell antigen is EphA2.

In some embodiments, the antigen binding domain is against a disease-associated antigen, selected from EphA2, EGFRviii, AFP, CEA, CA-125, MUC-1, CD123, CD30, SlamF7, CD33, EGFRvIII, BCMA, GD2, CD38, PSMA, B7H3, EPCAM, IL-13Ra2, PSCA, Mesothelin, Her2, CD19, CD20, CD22, sial-LewisA, LewisY, CIAX, or another tumor-enriched protein.

In some embodiments, the modified cell is capable of selectively engulfing tumor cells, cross-presenting a tumor antigen, and/or activating T-cells to respond to the tumor antigen.

In some embodiments, the modified cell is capable of cross-presenting tumor antigens (or having tumor antigen cross-presentation), wherein antigen cross-presentation is the ability of a cell to present internalized antigens on type I major histocompatibility complex molecules (MHC I), which is necessary for an efficient adaptive immune response against tumor cells.

In some embodiments, the modified cell is capable of eliminating antigen positive (Ag+) tumors targeted by the CARs, and indirectly eliminate Ag- solid tumor cells (not recognized by the CAR), through epitope spreading.

Another aspect of the present disclosure is a pharmaceutical composition comprising the modified cell described herein.

Another aspect of the present disclosure is a method of stimulating an adaptive antitumor T cell response in a subject comprising: administering to the subject a therapeutically effective amount of a pharmaceutical composition comprising a chimeric antigen receptor dendritic cell (CAR-DC); wherein, the CAR comprises an antigen binding domain, a transmembrane domain, and an intracellular domain; the intracellular domain comprises a FMS-like tyrosine kinase 3 (Flt3) signaling domain; and/or the cell is a dendritic cell or a progenitor cell thereof.

In some embodiments, the subject has a proliferative disease, disorder, or condition (e.g., cancer).

In some embodiments, the method induces phagocytosis of cancer cells in a subject.

In some embodiments, the CAR-DC cross-primes an anti-tumor T-cell response.

In some embodiments, the CAR-DC creates a tumor-eliminating immune response.

In some embodiments, the proliferative disease, disorder, or condition is a malignant tumor, solid tumor, or liquid tumor.

In some embodiments, the modified cell directly targets CAR-antigen positive (Ag+) tumor cells for elimination; or indirectly targets CAR-antigen negative (Ag-) tumor cells for elimination through cross-presentation and epitope spreading.

Another aspect of the present disclosure provides for a method of making a population of modified immune cells (e.g., DCs, cDC1s), comprising: (i) providing or having been provided a population of cells from a subject (e.g., mononuclear or stem cells from circulation, cord, or bone marrow); (ii) culturing the population of cells in a medium comprising an FMS-like tyrosine kinase 3 (Flt3) agonist for at least about one day; (iii) introducing a Flt3-based chimeric antigen receptor (CAR) into the cells from (ii); and/or (iv) culturing the cells from (iii) in a medium comprising an FMS-like tyrosine kinase 3 (Flt3) agonist for an amount of time sufficient to form a modified cell, wherein, the CAR comprises an antigen binding domain, a transmembrane domain, and an intracellular domain, the intracellular domain comprising a FMS-like tyrosine kinase 3 (Flt3) singling domain.

In some embodiments, the amount of time sufficient to form the modified cell is between about 2 days and about 15 days.

In some embodiments, introducing the CAR into the bone marrow cells comprises introducing an intracellular nucleic acid sequence encoding a protein product comprising an Flt3 or an Flt3-like intracellular domain into the cells.

In some embodiments, the modified cell is capable of antigen cross-presentation, an adaptive antitumor immune response, or activation of antitumor T cells.

In some embodiments, the modified cell is a dendritic cell or a conventional type 1 dendritic cell (cDC1).

Other objects and features will be in part apparent and in part pointed out hereinafter.

### BRIEF DESCRIPTION OF THE FIGURES

The application file contains at least one drawing executed in color. Copies of this patent application publication with color drawing(s) will be provided by the Office upon request and payment of the necessary fee.

FIGS. 1A-1E show CAR macrophages exert local tumor killing, but do not generate a clinically significant systemic anti-tumor response. FIG. 1A is a schematic showing mice were injected with syngeneic tumor in the bilateral flank, and once established, were injected with FcR CAR macrophages into one tumor. FIG. 1B shows a cartoon of a CAR expressing cell targeting and engulfing an Ag+ tumor cell. FIG. 1C is a graph showing tumor burden was measured over time in both the injected tumor. FIG. 1D is a graph showing tumor burden was measured over time in both the uninjected tumor. FIG. 1E is a graph showing one mouse exhibited a complete response at the site of injection; this mouse was re-injected with tumor to test for anti-tumor immunity.

FIG. 2 shows CAR Design: the CAR vector contains a signal peptide (SP) that drives surface expression, followed by a tumor antigen binding domain (here, an scFv from an antibody recognizing EphA2), followed by an extracellular domain (EC) and transmembrane domain (TM; here, derived from CD8), and an intracellular domain, followed by a P2A cleavage sequence and RFP to assess transduction efficiency. The intracellular domains vary by CAR in these experiments, and include the signaling domains from the Fc receptor (top), TLR4 (second), or Flt3 (third). The bottom construct is the Control CAR, which lacks an intracellular signaling domain.

FIG. 3 shows CAR Expression. Bone marrow cells were transduced with empty vector, an Fc receptor based CAR, a Flt3L based CAR, a TLR4 based CAR, or a control CAR lacking an intracellular domain. FACS analysis (top row) demonstrate CAR expression on the y-axis, and RFP expression on the x-axis. Fluorescence microscopy further confirms expression of the RFP transduction marker (bottom row).

FIG. 4A and FIG. 4B show Ova antigen-expressing syngeneic tumor was co-incubated with the indicated CAR for 12 hours, followed by the addition of CFSE-labeled OT1 T-cells. Three days later, antigen cross-presentation-induced T-cell proliferation was assessed by flow cytometry. FIG. 4A is a graph showing the quantification of T-cell proliferation. “Control CAR” is a non-signaling CAR lacking an intracellular domain. FIG. 4B are flow plots showing CFSE proliferation histograms of the CD3+ CD8+ T-cells. FIG. 4C shows CAR transduced bone marrow cells that were sorted for RFP positivity and incubated with zsGreen expressing tumor cells in a 1:1 ratio. Red/green double positive cells, indicating red transduced cells that had internalized green tumor, were automatically quantified by live video microscopy at indicated time points.

FIG. 5A and FIG. 5B show GFP+ Ova antigen-expressing syngeneic tumor was incubated with the indicated CAR in addition to OT1 T-cells in a 2:1:1 ratio, respectively. FIG. 5A is a graphs showing after ten days, tumor area was quantified. FIG. 5B shows individual wells are shown at low power magnification (2.5x); tumor is green.

FIG. 6A and FIG. 6B shows HoxB8 multipotent cells were transduced with a control non-signaling CAR, or the Flt3 CAR, sorted for CAR positivity, then cocultured with tumor cells in the absence of exogenous Flt3 ligand. FIG. 6A shows Live HoxB8 cells were quantified after two days of coculture. FIG. 6B shows representative images demonstrate uniform death of control CAR HoxB8 cells two days after the replacement of Flt3L with tumor, which Flt3 CAR HoxB8 cells survive and clump around tumor.

FIG. 7A and FIG. 7B show Flt3 based CARs improve generation of CAR-cDC1s. FIG. 7A shows bone marrow cells were transduced with the indicated CAR and differentiated into DCs. Percent CAR-transduced cDC1s were quantified by flow cytometry and compared with primary, non-CAR transduced DCs. FIG. 7B shows flow cytometry primary gating strategy shown, in which cDCs are B220-, CD11c+, MHC-II+, and cDC1 and cDC2s are further differentiated by CD24 and Sirpa positivity, respectively.

FIGS. 8A-8C show Flt3 CAR DCs induce a systemic anti-tumor adaptive response that eliminates local and distant sites of disease, and protects from tumor rechallenge. Sarcoma was orthotopically injected into the bilateral flank of syngeneic mice, and once established, one of the two tumors in each mouse was injected with control or Flt3 CAR DCs. FIG. 8A shows tumor burden was measured for treated tumors. FIG. 8B shows tumor burden was measured for untreated tumors. While all control CAR and untreated mice progressed bilaterally, Flt3 CAR DC-treated tumors slowly regressed bilaterally beginning 1-2 weeks following local treatment. FIG. 8C shows Flt3 CAR DC-treated mice exhibiting complete tumor response were re-injected with tumor, and tumor re-growth was not observed.

### DETAILED DESCRIPTION

The present disclosure is based, at least in part, on the discovery that dendritic cells that have been genetically modified to express a chimeric antigen receptor (CAR) are capable of targeting tumor cells for phagocytosis and cytotoxicity through T-cell cross-priming. As shown herein, CAR dendritic cells (CAR-DCs) can be used to treat various cancers and malignancies, including solid tumors. Previously described CAR macrophages (CAR-Ms) have not successfully cross-primed T-cells after phagocytosing or pinocytosing tumor cells, and have not been successful in eliminating solid tumors in vivo. The present disclosure describes a method of generating functional CAR-DCs, which selectively engulf tumor cells and cross-present endogenous tumor antigen in a manner that cross-primes tumor antigen-reactive T-cells. The disclosure demonstrates that CAR-DCs derived from a Flt3 based CAR are able to successfully generate cDC1 cells, whereas traditional Fc receptor based CARs introduced into myeloid precursor cells do not form cDC1s, but rather form macrophages, even when they are grown in the presence of the DC-differentiating cytokine Flt3L. The inability of non-Flt3-based CARs to successfully generate DCs appears to be due to basal signaling from the CAR that impairs proper differentiation to the cDC1 phenotype. The present disclosure thus provides compositions and methods for creating an adaptive immune response using CAR DCs. The adaptive immune response generated is useful to target and kill both CAR-Ag⁺ and CAR-Ag⁻ tumor or cancer cell, as well as create an immune memory which prevents tumor or cancer cell recurrence.

A composition of the disclosure may optionally comprise one or more additional drug or therapeutically active agent in addition to the CAR-DCs. A composition of the disclosure may further comprise a pharmaceutically acceptable excipient, carrier, or diluent. Further, a composition of the disclosure may contain preserving agents, solubilizing agents, stabilizing agents, wetting agents, emulsifiers, salts (substances of the present invention may themselves be provided in the form of a pharmaceutically acceptable salt), buffers, coating agents, or antioxidants.

Other aspects and iterations of the invention are described more thoroughly below.

### I. Compositions

For the first time, as described herein, is a CAR construct that allows for the generation of functional CAR-DCs, which selectively engulf tumor cells and cross-present endogenous tumor antigens in a manner that cross-primes endogenous tumor antigen-reactive T-cells to eliminate remaining tumor, in vitro and in vivo.

Previous work has described CAR-macrophages. Macrophages, like dendritic cells, can phagocytose material and can present antigens. However, in vivo, macrophages are unable to effectively cross-present tumor antigens, and are unable to create a tumor-eliminating immune response through T-cell cross-priming. In vivo, DCs, and particularly the subset of DCs known as cDC1s, are the only cells capable of tumor antigen cross-presentation and T-cell cross-priming; without cDC1s, an adaptive antitumor response is not achievable, an anti-tumor immune response cannot be mounted, and tumor cannot be eliminated by the immune system in vivo. Thus, conceptually, CAR-macrophages can achieve the goal of direct tumor phagocytosis or possibly direct cellular cytotoxicity. However, they cannot achieve the goal of antigen cross-presentation and cannot create an effective adaptive anti-tumor T cell response.

CAR-macrophages have been created by fusing the intracellular domain of various macrophage receptors that induce phagocytosis, such as fc receptors, toll-like receptors, or other macrophage or T-cell based receptors, with a tumor-recognizing scFv extracellular domain. No CARs to date have successfully been created that endow cells with DC capacity; that is, the ability to cross-prime an effective anti-tumor T-cell response, particularly in vivo.

### (A) CAR Dendritic Cells (CAR-DCs)

The present disclosure provides chimeric antigen receptor-bearing dendritic cell (CAR-DCs), pharmaceutical compositions comprising them, and methods of immunotherapy for the treatment of cancers or tumors. A CAR-DC is a dendritic cell which expresses a chimeric antigen receptor (CAR). As described herein, dendritic cells, or precursors or progenitors thereof, can be modified to form CAR dendritic cells (CAR-DCs). A chimeric antigen receptor (CAR), is a recombinant fusion protein comprising: 1) an extracellular ligand-binding domain, i.e., an antigen-recognition domain, 2) a transmembrane domain, and 3) a signaling transducing domain.

CAR-DCs comprising a Flt3-based CAR construct are able to functionally phagocytose tumor cells in a CAR-dependent manner and cross-prime anti-tumor T-cells by tumor uptake and antigen cross-presentation, whereas CAR-macrophages cannot. Thus, the term includes DCs that initiate an immune response and/or present an antigen to T lymphocytes and/or provide T-cells with any other activation signal required for stimulation of an adaptive immune response.

As described herein, CAR-DCs can be generated by exposing isolated dendritic cell progenitors, such as stem cells (pluripotent, multipotent, hematopoietic, or other stem cells), multipotent progenitors, common myeloid progenitors (CMP), myeloid dendritic cell progenitors (MDP), common dendritic cell progenitor (CDP), bone marrow mononuclear cells, peripheral blood mononuclear cells (PBMC), or splenocytes, to a DC proliferative stimulus such as Flt3L. The cell can then be transduced with the CAR of interest and further exposed to the DC differentiating factor Flt3L for an amount of time sufficient to generate dendritic cell-like cells (DC-like cells) prior to treatment. For example, the cell can be exposed to Flt3L for about 2 to 15 days to promote differentiation.

The present disclosure provides for modified dendritic cells (DCs) and modified precursors and modified progenitors of DCs. DCs are immune cells that are capable of antigen cross-presentation and are critical in initiating an adaptive immune response, particularly to tumor. Numerous studies demonstrate that DCs are limited in the tumor microenvironment and even in cancer patients in general. Further, even if DCs are present, they can induce tolerance or rejection of an antigen, or have no effect at all as they generally have no strong signal instructing them a tumor cell is foreign or threatening and in need of being eliminated.

A dendritic cell can be a subset of dendritic cells. As an example, a subset of DCs can be, for example, plasmacytoid DC (pDC), myeloid/conventional/classical DC1 (cDC1), myeloid/conventional/classical DC2 (cDC2), or monocyte-derived DC (moDC).

A progenitor can be any cell that is capable of differentiating into a DC. For example, a DC progenitor can be a stem cell (pluripotent, multipotent, hematopoietic, or other stem cell), multipotent progenitor, common myeloid progenitor (CMP), myeloid and dendritic cell progenitor (MDP), a lymphoid-primed multipotent progenitor (LMPP), common dendritic cell progenitor (CDP), bone marrow monocytes, a peripheral blood mononuclear cell (PBMC), or splenocytes.

A precursor of DCs can be a progenitor, as described above, or any cell that can be induced or reprogramed to differentiate into DCs, such as fibroblasts. For example, precursors to DCs can be stem cells, monocytes, myeloid precursor cells, myeloid-derived precursor cells, peripheral blood mononuclear cells (PBMCs), or bone marrow monocytes (BMM).

CAR-DCs can be autologous, meaning that they are engineered from a subject’s own cells, or allogeneic, meaning that the cells are sourced from a healthy donor, and in many cases, engineered so as not to provoke a host-vs-graft or graft-vs-host reaction. Donor cells may also be sourced from cord blood or generated from induced pluripotent stem cells.

The present disclosure provides for modified conventional type I dendritic cells (cDC1s), which can be generated by differentiating CAR-DCs. As described herein, in vivo, DCs, and particularly the subset of DCs known as cDC1s, are the only immune cells capable of effective tumor antigen cross-priming. Antigen cross-priming refers to the stimulation of antigen-specific naïve cytotoxic CD8 T-cells into activated cytotoxic CD8 T-cells by antigen presenting cells that have acquired and cross-presented extracellular antigen, in this case acquired from tumor. Antigen cross-presentation refers to the ability of a cell to present internalized antigens on type I major histocompatibility complex molecules (MHC I). Antigen cross-presentation and cross-priming are known to be necessary for an efficient adaptive immune response against tumor cells.

Without cDC1s, an adaptive antitumor response is not achievable, an anti-tumor immune response cannot be mounted, and tumor cannot be eliminated by the immune system in vivo, see, e.g. the below Examples.

As described herein, transducing a dendritic cell or precursor thereof with a Flt3-based CAR is uniquely able to produce true, programmable and functional cDC1s, which has not been before demonstrated. Traditional Fc receptor-based CARs introduced into myeloid precursor cells do not form cDC1s, but rather form macrophages or cDC2s, even though they are grown in the cDC1-differentiating cytokine Flt3L. The inability of non-Flt3-based CARs to successfully generate DCs appears to be due to basal signaling from the CAR that impairs proper differentiation to the cDC1 phenotype. Thus, Flt3-based CARs can be called “CAR-DCs,” which are to be distinguished from other CARs (based on the Fc receptor or other inflammatory or macrophage receptor domains), which when expressed in progenitor cells produce CAR- macrophages (CAR-Ms) that possess significantly inferior ability to cross-prime T-cells. Unlike CAR-Ms or cDC1s, CAR-DCs have superior ability to selectively engulf tumor cells, cross-present tumor antigen, and activate T-cells to respond to the tumor antigen.

As described herein, cDC1s can be identified based on flow cytometry for specific surface protein expression signatures, and confirmed by their functional capacity to cross-prime T-cells against engulfed cell-associated antigen. For example, the cDC surface expression profile can be lineage-negative B220⁻, CD11c⁺, and MHC-II⁺, and cDC1 and cDC2s can be further differentiated by CD24 and Sirpa expression.

### (B) Flt3-Based CAR Constructs

CAR designs are generally tailored to each cell type. The present disclosure is drawn to dendritic cells, but could be useful in other immune cell types. Disclosed herein are dendritic cells engineered to express chimeric antigen receptors (CARs).

CARs are designed in a modular fashion that comprise an extracellular target-binding domain (e.g., antigen-binding domain, tumor binding domain), a hinge region, a transmembrane domain that anchors the CAR to the cell membrane, and one or more intracellular domains that transmit activation signals. A chimeric antigen receptor (CAR) of the present disclosure comprises a signal transducing domain or intracellular signaling domain of a CAR which is responsible for intracellular signaling following the binding of the extracellular ligand binding domain to the target resulting in the activation of the immune cell and immune response. In other words, the signal transducing domain is responsible for the activation of at least one of the normal effector functions of the immune cell in which the CAR is expressed. For example, the effector function of a dendritic cell can be increased survival, differentiation, phagocytosis, and/or antigen cross-presentation. Thus, the term “signal transducing domain” refers to the portion of a protein which transduces the effector signal function signal and directs the cell to perform a specialized function. In the case of CAR T-cells, depending on the number of costimulatory domains, CARs can be classified into first (CD3z only), second (one costimulatory domain + CD3z), or third generation CARs (more than one costimulatory domain + CD3z). Costimulatory domains utilized in the present CAR DC may similarly be used to increase or decrease the cell’s function, persistence, or proliferation. These domains may include, but are not limited to, domains derived from an Fc Receptor, a TLR, CSF1R, CD40, PD-1, 41BB, CD28, OX40, ICOS, SR-A1, SR-A2, SR-CL2, SR-C, SR-E, MARCO, dectin 1, DEC-205, DEC-206, DC-SIGN, or other proteins with signaling functions. Introduction of CAR molecules into a DC successfully redirects the DC with additional antigen specificity and provides the necessary signals to drive full DC activation and function.

In one embodiment, the nucleic acid sequence encodes a CAR with an intracellular signaling component comprising at least 50% sequence identity, at least 60% sequence identity, at least 70% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to the intracellular domain from the protein Flt3. FMS-like tyrosine kinase 3 (FLT-3) (also known as cluster of differentiation antigen 135 (CD135), receptor-type tyrosine-protein kinase Flt3, or fetal liver kinase-2 (Flk2)) is a protein that in humans is encoded by the FLT3 gene. Flt3 is a cytokine receptor which belongs to the receptor tyrosine kinase class III. Flt3 is the receptor for the cytokine Flt3 ligand (FLT3L). Flt3 is composed of five extracellular immunoglobulin-like domains, an extracellular domain, a transmembrane domain, a juxtamembrane domain and a tyrosine-kinase domain consisting of 2 lobes that are connected by a tyrosine-kinase insert. Cytoplasmic Flt3 undergoes glycosylation, which promotes localization of the receptor to the membrane. The nucleic acid sequences and peptide sequences can be found in publicly available databases, including, for example Entrez gene accession number 2322 and UniProt accession number P36888.

Ftl3 tyrosine-protein kinase that acts as cell-surface receptor for the cytokine FLT3L and regulates differentiation, proliferation and survival of hematopoietic progenitor cells and of dendritic cells. Flt3 promotes phosphorylation of SHC1 and AKT1, and activation of the downstream effector MTOR. It promotes activation of RAS signaling and phosphorylation of downstream kinases, including MAPK1/ERK2 and/or MAPK3/ERK1. It also has been shown to promote phosphorylation of FES, FER, PTPN6/SHP, PTPN11/SHP-2, PLCG1, and STAT5A and/or STAT5B. Activation of wild-type FLT3 causes only marginal activation of STAT5A or STAT5B.

In one embodiment, the composition of the CAR-DC is: Signal Peptide - Target binding domain - hinge domain - transmembrane domain - Flt3 intracellular domain.

The Flt3 intracellular domain was discovered to be critical for effective CAR-DC generation. SEQ ID NO: 1 is an example of a human Flt3 domain:

HKYKKQFRYESQLQMVQVTGSSDNEYFYVDFREYEYDLKWEFPRENLEFG
KVLGSGAFGKVMNATAYGISKTGVSIQVAVKMLKEKADSSEREALMSELK
MMTQLGSHENIVNLLGACTLSGPIYLIFEYCCYGDLLNYLRSKREKFHRT
WTEIFKEHNFSFYPTFQSHPNSSMPGSREVQIHPDSDQISGLHGNSFHSE
DEIEYENQKRLEEEEDLNVLTFEDLLCFAYQVAKGMEFLEFKSCVHRDLA
ARNVLVTHGKVVKICDFGLARDIMSDSNYVVRGNARLPVKWMAPESLFEG
IYTIKSDVWSYGILLWEIFSLGVNPYPGIPVDANFYKLIQNGFKMDQPFY
ATEEIYIIMQSCWAFDSRKRPSFPNLTSFLGCQLADAEEAMYQNVDGRVS
ECPHTYQNRRPFSREMDLGLLSPQAQVEDS.

Another, non-limiting example of a Flt3 domain useful in the present invention is a mouse Flt3 domain:

HKYKKQFRYESQLQMIQVTGPLDNEYFYVDFRDYEYDLKWEFPRENLEFG
KVLGSGAFGRVMNATAYGISKTGVSIQVAVKMLKEKADSCEKEALMSELK
MMTHLGHHDNIVNLLGACTLSGPVYLIFEYCCYGDLLNYLRSKREKFHRT
WTEIFKEHNFSFYPTFQAHSNSSMPGSREVQLHPPLDQLSGFNGNSIHSE
DEIEYENQKRLAEEEEEDLNVLTFEDLLCFAYQVAKGMEFLEFKSCVHRD
LAARNVLVTHGKVVKICDFGLARDILSDSSYVVRGNARLPVKWMAPESLF
EGIYTIKSDVWSYGILLWEIFSLGVNPYPGIPVDANFYKLIQSGFKMEQP
FYATEGIYFVMQSCWAFDSRKRPSFPNLTSFLGCQLAEAEEAMYQNMGGN
VPEHPSIYQNRRPLSREAGSEPPSPQAQ.

In some embodiments, the Flt3 domain used has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, at least 99% sequence identity with SEQ ID NO:1 or SEQ ID NO:2.

Furthermore, the CAR construct moieties or components can be operably linked with a linker. A linker can be any nucleotide sequence capable of linking the moieties described herein. For example, the linker can be any amino acid sequence suitable for this purpose (e.g., of a length of 8 - 80 amino acids, depending on the target-binding domain being used).

Various intracellular domains have different functions in different cell types. The present disclosure provides for an intracellular signaling domain useful in DCs. As described herein, Fc receptor-based, toll-like receptor (TLR)-based, or FMS-like tyrosine kinase 3 (Flt3)-based IC domains were directly compared, and the Flt3-based IC domains were discovered to be the most effective at generating functional CAR-DCs.

The FMS-like tyrosine kinase 3 (Flt3)-based IC domain can be any Flt3-based or Flt3-derived IC domain, such as active variants or functional fragments of the human Flt3 IC domain of SEQ ID NO: 1 or SEQ ID NO: 2.

As described herein, the intracellular domain can be a FMS-like tyrosine kinase 3 (Flt3) intracellular domain. The Flt3 signaling domain is derived from the Flt3 gene. Flt3 encodes a class III receptor tyrosine kinase that acts as a receptor for the cytokine Flt3 ligand (Flt3L). The intracellular domain derived from Flt3 was shown to be critical for successfully achieving CAR dendritic cells.

In some embodiments, the CAR-DCs can join the properties of different intracellular domains in one single dendritic cell by combining two or more intracellular domains in a CAR. For example, such combinations can include one intracellular domain from the Flt3 family and one intracellular domain from an ITAM domain-containing protein or a TIR-domain containing protein, resulting in the simultaneous activation of different signaling pathways. These are considered costimulatory domains, described in more detail above.

Each costimulatory domain can have unique properties. Differences in the affinity of the scFv, the intensity of antigen expression, the probability of off-tumor toxicity, or the disease to be treated may influence the selection of the intracellular domain.

As described herein, the CAR can comprise an antigen binding domain or tumor binding domain. The antigen binding domain can comprise any domain that binds to an antigen expressed by the targeted cell type (e.g., an antigen expressed by a tumor cell) or a fragment thereof (see e.g., Saar Gill et al. U.S. App. No. 15/747,555 incorporated herein by reference in its entirety). For example, the antigen binding domain can be an antibody (from human, mouse, or other animal), a humanized antibody, a monoclonal antibody, a polyclonal antibody, a synthetic antibody, a camelid antibody, a native receptor or ligand, or a fragment thereof. For example, the antigen binding domain can be a single-chain variable fragment (scFv) of an antibody. The antigen binding domain can be directed to various tumor associated proteins, which may include EphA2, alphafetoprotein (AFP), carcinoembryonic antigen (CEA), CA-125, MUC-1 antibody, CD19, CD20, CD123, CD22, CD30, SlamF7, CD33, EGFRvIII, BCMA, GD2, CD38, PSMA, B7H3, EPCAM, IL-13Ra2, PSCA, Mesothelin, Her2, LewisY, LewisA, CIAX, epithelial tumor antigen (ETA), tyrosinase, melanoma-associated antigen (MAGE), abnormal products of ras or p53, or other proteins found to be more highly enriched on the surface of tumor cells than critical normal tissues. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized. Non-limiting examples of tumor antigens include carbonic anhydrase IX (CAIX), carcinoembryonic antigen (CEA), CD8, CD7, CD10, CD19, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), and Wilms tumor protein (WT-1), BCMA, NKCS1, EGF1R, EGFR-VIII, CD99, CD70, ADGRE2, CCR1, LILRB2, PRAME CCR4, CD5, CD3, TRBC1, TRBC2, TIM-3, Integrin B7, ICAM-1, CD70, Tim3, CLEC12A and ERBB.

Targeting antibody fragments or scFvs, as described herein, can be against any disease-associated antigen or tumor-associated antigen (TAA). A TAA can be any antigen known in the art to be associated with tumors.

scFvs are well known in the art to be used as a binding moiety in a variety of constructs (see e.g., Sentman 2014 Cancer J. 20 156-159; Guedan 2019 Mol Ther Methods Clin Dev. 12 145-156). Any scFv known in the art or generated against an antigen using means known in the art can be used as the binding moiety.

The antigen-binding capability of the CAR is defined by the extracellular scFv. The format of a scFv is generally two variable domains linked by a flexible peptide sequence, either in the orientation VH-linker-VL or VL-linker-VH. The orientation of the variable domains within the scFv, depending on the structure of the scFv, may contribute to whether a CAR will be expressed on the dendritic cell surface or whether the CAR-DCs target the antigen and signal. In addition, the length and/or composition of the variable domain linker can contribute to the stability or affinity of the scFv.

The scFv, a critical component of a CAR molecule, can be carefully designed and manipulated to influence specificity and differential targeting of tumors versus normal tissues.

Typically, the extracellular ligand-binding domain is linked to the signaling transducing domain of the chimeric antigen receptor (CAR) by a transmembrane domain (Tm). The transmembrane domain traverses the cell membrane, anchors the CAR to the DC surface, and connects the extracellular ligand-binding domain to the signaling transducing domain, impacting the expression of the CAR on the DC surface. The distinguishing feature of the transmembrane domain in the present disclosure is the ability to be expressed at the surface of a DC to direct an immune cell response against a pre-defined target cell. The transmembrane domain can be derived from natural or synthetic sources. Alternatively, the transmembrane domain of the present disclosure may be derived from any membrane-bound or transmembrane protein.

Non-limiting examples of transmembrane polypeptides of the present disclosure include CD8 alpha or beta, alpha, beta or zeta chain of the T-cell receptor, CD28, CD3 epsilon, CD45, CD4, CDS, CD9, CD16, CD22, CD33, CD37, CD64, CDS0, CD86, CD134, CD137 and CD154. Alternatively, the transmembrane domain can be synthetic and comprise predominantly hydrophobic amino acid residues (e.g., leucine and valine).

The transmembrane domain can further comprise a hinge region between extracellular ligand-binding domain and said transmembrane domain. The term “hinge region” generally means any oligo- or polypeptide that functions to link the transmembrane domain to the extracellular ligand-binding domain. In particular, hinge region is used to provide more flexibility and accessibility for the extracellular ligand-binding domain. A hinge region may comprise up to 300 amino acids, preferably 5 to 100 amino acids and most preferably 8 to 50 amino acids. Hinge region may be derived from all or parts of naturally-occurring molecules such as CD28, 4-1BB (CD137), OX-40 (CD134), CD3ζ, the T cell receptor α or β chain, CD45, CD4, CD5, CD8b, CD8α, CD9, CD16, CD22, CD33, CD37, CD64, CD80, CD86, ICOS, CD154 or from all or parts of an antibody constant region. Alternatively, the hinge region may be a synthetic sequence that corresponds to a naturally-occurring hinge sequence or the hinge region may be an entirely synthetic hinge sequence. In one embodiment, the hinge domain comprises a part of human CD8α, FcγRIIIα receptor, or IgGl, and have at least 80%, 90%, 95%, 97%, or 99% sequence identity thereto.

The hinge, also referred to as a spacer, is in the extracellular structural region of the CAR that separates the binding units from the transmembrane domain. The hinge can be any moiety capable of ensuring proximity of the dendritic cell to the target. The hinge can be any moiety capable of ensuring proximity of the DC to the target (e.g., CD8-based hinge). With the exception of CARs based on the entire extracellular moiety of a receptor, the majority of CAR (such as CAR T) cells are designed with immunoglobulin (Ig)-like domain hinges or CD8 hinges, but any protein sequence that proves a space between the transmembrane domain and target-binding domain may function as an effective hinge.

Hinges generally supply stability for efficient CAR expression and activity. The hinge (also in combination with the transmembrane domain), also ensures proper proximity to target.

The hinge also provides flexibility to access the targeted antigen. The optimal spacer length of a given CAR can depend on the position of the targeted epitope. Long spacers can provide extra flexibility to the CAR and allow for better access to membrane-proximal epitopes or complex glycosylated antigens. CARs bearing short hinges can be more effective at binding membrane-distal epitopes. The length of the spacer can be important to provide adequate intercellular distance for immunological synapse formation. As such, hinges may be optimized for individual epitopes accordingly.

Here, the hinge can be operably linked to the transmembrane domain.

Optionally, an extracellular signaling domain can be incorporated into the CAR construct to propagate signaling. The extracellular signaling domain can be cloned into the hinge region, but can be chosen based on the target.

A signal peptide directs the transport of a secreted or transmembrane protein to the cell membrane and/or cell surface to allow for correct localization of the polypeptide. Particularly, the signal peptide of the present disclosure directs the appended polypeptide, i.e., the CAR receptor, to the cell membrane wherein the extracellular ligand-binding domain of the appended polypeptide is displayed on the cell surface, the transmembrane domain of the appended polypeptide spans the cell membrane, and the signaling transducing domain of the appended polypeptide is in the cytoplasmic portion of the cell. In one embodiment, the signal peptide is the signal peptide from human CD8α. A functional fragment is defined as a fragment of at least 10 amino acids of the CD8α signal peptide that directs the appended polypeptide to the cell membrane and/or cell surface.

The CAR-DCs of the present disclosure may comprise one or more distinct CAR constructs. For example, a dual CAR-DC may be generated by cloning a protein encoding sequence of a first extracellular ligand-binding domain into a viral vector containing one or more costimulatory domains and a signaling transducing domain and cloning a second protein encoding sequence of a second extracellular ligand-binding domain into the same viral vector containing an additional one or more costimulatory domains and a signaling transducing domain resulting in a plasmid from which the two CAR constructs are expressed from the same vector. A tandem CAR-DC, is a DC with a single chimeric antigen polypeptide comprising two distinct extracellular ligand-binding domains capable of interacting with two different cell surface molecules, wherein the extracellular ligand-binding domains are linked together by a flexible linker and share one or more costimulatory domains, wherein the binding of the first or the second extracellular ligand-binding domain will signal through one or more the costimulatory domains and a signaling transducing domain.

Genetic modification of a DC or progenitor thereof can be accomplished by transducing a substantially homogeneous cell composition with a recombinant DNA construct. In certain embodiments, a retroviral vector (either gamma-retroviral or lentiviral) is employed for the introduction of the DNA construct into the cell. For example, a polynucleotide encoding a CAR can be cloned into a retroviral vector and expression can be driven from its endogenous promoter, from the retroviral long terminal repeat, or from a promoter specific for a target cell type of interest. Other viral vectors, or non-viral vectors may be used as well.

For initial genetic modification of a DC or progenitor thereof to include a CAR, a retroviral vector is generally employed for transduction, however any other suitable viral vector or non-viral delivery system can be used. The CAR can be constructed with an auxiliary molecule (e.g., a cytokine) in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). In certain embodiments, any vector or CAR disclosed herein can comprise a P2A peptide. Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Nat. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J Clin. Invest. 89:1817.

Other transducing viral vectors can be used to modify a DC or progenitor thereof. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of a DC or progenitor thereof. For example, a nucleic acid molecule can be introduced into a DC or progenitor thereof administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA’s and the tracrRNA can be packaged together to forma single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease Fokl. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome.cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

Any targeted genome editing methods can be used to place presently disclosed CARs at one or more endogenous gene loci of a presently disclosed immunoresponsive cell. In certain embodiments, a CRISPR system is used to deliver presently disclosed CARs to one or more endogenous gene loci of a presently disclosed immunoresponsive cell. In certain embodiments, zinc-finger nucleases are used to deliver presently disclosed CARs to one or more endogenous gene loci of a presently disclosed immunoresponsive cell. In certain embodiments, a TALEN system is used to deliver presently disclosed CARs to one or more endogenous gene loci of a presently disclosed immunoresponsive cell.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

Placement of a presently disclosed CAR can be made at any endogenous gene locus.

The present disclosure also provides pharmaceutical compositions. The pharmaceutical composition comprises a plurality of CAR-DCs, as an active component, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

Compositions comprising the presently disclosed CAR-DCs can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the CAR-DCs in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON’S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the CAR-DCs or their progenitors.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

The quantity of cells to be administered will vary for the subject being treated. In a one embodiment, between about 10³ and about 10¹⁰, between about 10⁵ and about 10⁹, or between about 10⁶ and about 10⁸ of the presently disclosed CAR-DCs are administered to a human subject. More effective cells may be administered in even smaller numbers. In certain embodiments, at least about 1×10⁸, about 2×10⁸, about 3×10⁸, about 4×10⁸, or about 5×10⁸ of the presently disclosed CAR-DCs are administered to a human subject. In certain embodiments, between about 1×10⁷ and 5×10⁸ of the presently disclosed CAR-DCs are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD₅₀ in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

Compositions comprising the presently disclosed CAR-DCs can be provided systemically or directly to a subject for inducing and/or enhancing an immune response to an antigen and/or treating and/or preventing a neoplasm, pathogen infection, or infectious disease. In certain embodiments, the presently disclosed CAR-DCs or compositions comprising thereof are directly injected into a tumor or organ of interest (e.g., an organ affected by a neoplasia). Alternatively, the presently disclosed CAR-DCs or compositions comprising thereof are provided indirectly to the organ of interest, for example, by administration into the circulatory system (e.g., the tumor vasculature). Expansion and differentiation agents can be provided prior to, during or after administration of the cells or compositions to increase production of T cells, NK cells, or CTL cells in vitro or in vivo.

The presently disclosed CAR-DCs can be administered in any physiologically acceptable vehicle, normally intravascularly, although they may also be introduced into bone or other convenient site where the cells may find an appropriate site for regeneration and differentiation (e.g., lymphatics). Usually, at least a population of about 1×10⁵ cells will be administered. The presently disclosed CAR-DCs can comprise a purified population of cells. Those skilled in the art can readily determine the percentage of the presently CAR-DCs in a population using various well-known methods, such as fluorescence activated cell sorting (FACS). Suitable ranges of purity in populations comprising the presently disclosed CAR-DCs are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

The presently disclosed compositions can be pharmaceutical compositions comprising the presently disclosed CAR-DCs or their progenitors and a pharmaceutically acceptable carrier. Administration can be autologous or heterologous. For example, CAR-DCs, or progenitors can be obtained from one subject, and administered to the same subject or a different, compatible subject. Peripheral blood derived CAR-DCs or their progeny (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed CAR-DCs), it can be formulated in a unit dosage injectable form (solution, suspension, emulsion).

### II. Methods

Cells disclosed herein, and/or generated using the methods disclosed herein, may be used in immunotherapy and adoptive cell transfer, for the treatment, or the manufacture of a medicament for treatment, of cancers, autoimmune diseases, infectious diseases, and other conditions. One aspect of the present disclosure provides for modified dendritic cells that stimulate an adaptive antitumor T cell response.

As described herein, the adaptive antitumor T cell response can be initiated or enhanced by antigen cross-presentation or cross-priming from the CAR-DCs. Cross-presentation describes the process in which the modified dendritic cells take up, process, and present antigens (e.g., a tumor cell antigen) on the surface of the cell on a complex with a MHC I molecule. The antigen is then recognized by a T cell. Cross-priming describes the process in which recognition of the antigen by the T cell results in the T cell becoming activated. The activated T cell is then capable of enhanced proliferation, persistence, and/or targeted, enhanced cytotoxicity towards tumor cells expressing that antigen.

As described herein, the adaptive antitumor T cell response can comprise, in a non-limiting example, an increase in T cell function. For example, T cell function can be assessed by the cytotoxic T cell lymphocyte assay (CTL), where an escalating ratio of effector T cells is mixed with target tumor cells for a defined amount of time (generally 4 hours), and tumor cell killing is quantified by tumor luciferase activity.

As described herein, the adaptive antitumor T cell response can also comprise an increase in T cell activation or proliferation. For example, T cell activation or proliferation can be measured by assessing CD4 and CD8 T cell division by FACS analysis for proliferation or for markers of activation, such as cytokine release.

As described herein, a successful adaptive antitumor T cell response can result in tumor cell cytotoxicity, further tumor cell phagocytosis, and reduction in tumor volume. The antitumor T cell response can directly eliminate antigen positive (Ag⁺) tumors targeted by the CARs, and indirectly eliminate CAR-Ag⁻ tumor cells (not recognized directly by the CAR), through cross-presentation and epitope spreading. Epitope spreading refers to the broadening of the immune response to include T cell and antibody specificities beyond the antigen that originally triggered the immune response. For example, epitope spreading can result in tumor cells that do not express the antigen targeted by the CAR to be targeted by T cells.

Thus, the present disclosure provides a method of stimulating an adaptive antitumor T cell response in a subject, wherein the method generally comprises administering an effective amount of CAR-DCs to the subject. The CAR-DCs targets a tumor or cancer cell, phagocytizes the tumor or cancer cell and cross-presents tumor antigens to the subject’s T cells. Accordingly, the CAR-DCs directly target antigen positive (Ag⁺) tumor or cancer cells for elimination and/or indirectly targets CAR-antigen negative (Ag⁻) tumor or cancer cells for elimination through cross-presentation and epitope spreading.

In another embodiment, the present disclosure provides methods for reducing or preventing cancer recurrence in a subject, wherein the method generally comprises administering an effective amount of CAR-DCs to the subject, which target an antigen expressed by the cancer or tumor cell. A recurrence occurs when the cancer comes back after the initial treatment. This can happen weeks, months, or even years after the primary or original cancer was treated. As described herein, the present disclosure is shown to produce a lasting adaptive antitumor T cell response, see, e.g., Example 1(vi).

In some embodiments, the present disclosure provides methods for treating a cancer or tumor in a subject, wherein the method generally comprises administering an effective amount of CAR-DCs to the subject, which target an antigen expressed by the cancer or tumor cell. This method of treatment can be particularly efficacious for solid tumors, but may be directed against any form of cancer. Traditional chimeric antigen receptor (CAR) T cells exhibit only a 1% complete response in solid tumors in clinical trials thus far. Solid tumors escape CAR T recognition if not all cells express the target antigen. Successfully creating an adaptive immune response in patients would overcome the failures of both types of immunotherapy. Dendritic cells (DCs) are critical in initiating an adaptive immune response. CAR-DCs enable new therapeutic strategies to directly eliminate antigen positive (Ag+) tumors targeted by the CARs, and indirectly eliminate Ag- solid tumor cells (not recognized by the CAR), through epitope spreading.

A tumor or cancer can be any tumor or cancer that occurs in (or is a metastatic cancer originating from) the bladder, breast, bone, cervix, muscle, brain and nervous system, endocrine system, endometrium, eye, lip, oral, liver, lung, gastrointestinal system (e.g., colon, rectal), genitourinary and gynecologic systems (e.g., cervix, ovary), head and neck, hematopoetic system, kidney, skin, pancreas, prostate, thyroid, bone, thoracic and respiratory system, or any other human tissue that has undergone a malignant transformation. A solid tumor is one derived from any human cell other blood cells.

The cancer may be a hematologic malignancy or solid tumor. Hematologic malignancies include leukemias, lymphomas, multiple myeloma, and subtypes thereof. Lymphomas can be classified various ways, often based on the underlying type of malignant cell, including Hodgkin’s lymphoma (often cancers of Reed-Sternberg cells, but also sometimes originating in B cells; all other lymphomas are non-Hodgkin’s lymphomas), B-cell lymphomas, T-cell lymphomas, mantle cell lymphomas, Burkitt’s lymphoma, follicular lymphoma, and others as defined herein and known in the art.

B-cell lymphomas include, but are not limited to, diffuse large B-cell lymphoma (DLBCL), chronic lymphocytic leukemia (CLL) /small lymphocytic lymphoma (SLL), and others as defined herein and known in the art.

T-cell lymphomas include T-cell acute lymphoblastic leukemia/lymphoma (T-ALL),, peripheral T-cell lymphoma (PTCL), T-cell chronic lymphocytic leukemia (T-CLL)Sezary syndrome, and others as defined herein and known in the art.

Leukemias include Acute myeloid (or myelogenous) leukemia (AML), chronic myeloid (or myelogenous) leukemia (CML), acute lymphocytic (or lymphoblastic) leukemia (ALL), chronic lymphocytic leukemia (CLL) hairy cell leukemia (sometimes classified as a lymphoma), and others as defined herein and known in the art.

Plasma cell malignancies include lymphoplasmacytic lymphoma, plasmacytoma, and multiple myeloma.

In some embodiments, the medicament can be used for treating cancer in a patient, particularly for the treatment of solid tumors such as melanomas, neuroblastomas, gliomas or carcinomas such as tumors of the brain, head and neck, breast, lung (e.g., non-small cell lung cancer, NSCLC), reproductive tract (e.g., ovary), upper digestive tract, pancreas, liver, renal system (e.g., kidneys), bladder, prostate and colorectum.

In another embodiment, the medicament can be used for treating cancer in a patient, particularly for the treatment of hematologic malignancies selected from multiple myeloma and acute myeloid leukemia (AML) and for T-cell malignancies selected from T-cell acute lymphoblastic leukemia (T-ALL), non-Hodgkin’s lymphoma, and T-cell chronic lymphocytic leukemia (T-CLL).

Non-limiting examples of neoplasms or cancers that may be treated with a method of the disclosure may include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing’s sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sézary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-cell lymphoma (cutaneous), T-cell leukemia and lymphoma, testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, or Wilms tumor (childhood).

Thus, aspects of the present disclosure is a method for treating a subject in need thereof. The terms “treat,” “treating,” or “treatment” as used herein, refers to the provision of medical care by a trained and licensed professional to a subject in need thereof. The medical care may be a diagnostic test, a therapeutic treatment, and/or a prophylactic or preventative measure. The object of therapeutic and prophylactic treatments is to prevent or slow down (lessen) an undesired physiological change or disease/disorder. Beneficial or desired clinical results of therapeutic or prophylactic treatments include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented.

Also provided is a process of treating or preventing a proliferative disease, disorder, or condition (e.g., a tumor or cancer, or metastases thereof) in a subject in need of administration of a therapeutically effective amount of a dendritic cell-based therapy as described herein so as to reduce or eliminate the tumor or cancer.

Methods described herein are generally performed on a subject in need thereof. A subject in need of the therapeutic methods described herein can be a subject having, diagnosed with, suspected of having, or at risk for developing a cancer or proliferative disease, disorder, or condition. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of CAR-DC therapy is, for example, that amount that would cause the desired therapeutic effect in a subject while minimizing undesired side effects. In various embodiments, an effective amount of a dendritic cell-based therapy described herein can substantially inhibit tumor growth or cancer progression, slow the progress of a tumor or cancer, or limit the development of a tumor or cancer.

When used in the treatments described herein, a therapeutically effective amount of a CAR-DC therapy can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to reduce or cure a proliferative disease, disorder, or condition.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of CAR-DC therapy can occur as a single event or over a time course of treatment. For example, dendritic cell-based therapy can be administered daily, weekly, bi-weekly, or monthly. For more chronic conditions, treatment could extend from several weeks to several months or years.

Treatment in accord with the methods described herein can be performed prior to, concurrent with, or after conventional treatment modalities for cancer or proliferative disease, disorder, or condition.

A CAR-DC therapy can be administered simultaneously or sequentially with another agent, such as an anti-cancer therapy, or another agent. For example, a dendritic cell-based therapy can be administered before, after, or simultaneously with another agent, such as a chemotherapeutic agent, another form of immune therapy, or radiation therapy. Simultaneous administration can occur through the administration of separate compositions, each containing one or more of a dendritic cell-based therapy and another agent, such as a chemotherapeutic agent, additional immune therapy, or radiation therapy. Simultaneous administration can occur through the administration of one composition containing two or more of a dendritic cell-based therapy, an antibiotic, an anti-inflammatory, or another agent, such as a chemotherapeutic agent, immune therapy, or radiation therapy.

The administration of CAR-DCs or a population of CAR-DCs of the present disclosure of the present disclosure be carried out by aerosol inhalation, injection, ingestion, transfusion, implantation or transplantation. The CAR-DCs compositions described herein, i.e., mono CAR, dual CAR, tandem CARs, may be administered to a patient subcutaneously, intradermally, intratumorally, intranodally, intramedullary, intramuscularly, by intravenous or intralymphatic injection, or intraperitoneally. In one embodiment, the cell compositions of the present disclosure are preferably administered by intravenous injection.

As noted above, the administration of CAR-DCs cells or a population of CAR-DCs can consist of the administration of 10³-10⁹ cells per kg body weight, preferably 10⁵ to 10⁶ cells/kg body weight including all integer values of cell numbers within those ranges. The CAR-DCs or a population of CAR-DCs can be administrated in one or more doses. In another embodiment, the effective amount of CAR-DCs or a population of CAR-DCs are administrated as a single dose. In another embodiment, the effective amount of cells are administered as more than one dose over a period time. Timing of administration is within the judgment of a health care provider and depends on the clinical condition of the patient. The CAR-DCs or a population of CAR-DCs may be obtained from any source, such as a blood bank or a donor. While the needs of a patient vary, determination of optimal ranges of effective amounts of a given CAR-DCs population(s) for a particular disease or conditions are within the skill of the art. An effective amount means an amount which provides a therapeutic or prophylactic benefit. The dosage administered will be dependent upon the age, health and weight of the patient recipient, type of concurrent treatment, if any, frequency of treatment, and the nature of the effect desired.

In another embodiment, the effective amount of CAR-DCs or a population of CAR-DCs or composition comprising those CAR-DCs are administered parenterally. The administration can be an intravenous administration. The administration of CAR-DCs or a population of CAR-DCs or composition comprising those CAR-DCs can be directly done by injection within a tumor.

In one embodiment of the present disclosure, the CAR-DCs or a population of the CAR-DCs are administered to a patient in conjunction with, e.g., before, simultaneously or following, any number of relevant treatment modalities, including but not limited to, treatment with cytokines, or expression of cytokines from within the CAR-DCs, that enhance dendritic cell or T-cell proliferation and persistence and, include but not limited to, Flt3L, IL-2, IL-7, and IL-15 or analogues thereof.

In some embodiments, the CAR-DCs or a population of CAR-DCs of the present disclosure may be used in combination with agents that inhibit immunosuppressive pathways, including but not limited to, inhibitors of TGFβ, interleukin 10 (IL-10), adenosine, VEGF, indoleamine 2,3 dioxygenase 1 (IDO1), indoleamine 2,3-dioxygenase 2 (IDO2), tryptophan 2-3-dioxygenase (TDO), lactate, hypoxia, arginase, and prostaglandin E2.

In another embodiment, the CAR-DCs or a population of CAR-DCs of the present disclosure may be used in combination with T-cell checkpoint inhibitors, including but not limited to, anti-CTLA4 (such as Ipilimumab) anti-PD1 (such as Pembrolizumab, Nivolumab, Cemiplimab), anti-PDL1 (such as Atezolizumab, Avelumab, Durvalumab), anti-PDL2, anti-BTLA, anti-LAG3, anti-TIM3, anti-VISTA, anti-TIGIT, and anti-KIR.

In another embodiment, the CAR-DCs or a population of CAR-DCs of the present disclosure may be used in combination with T cell agonists, including but not limited to, antibodies that stimulate CD28, ICOS, OX-40, CD27, 4-1BB, CD137, GITR, and HVEM.

In another embodiment, the CAR-DCs or a population of CAR-DCs of the present disclosure may be used in combination with therapeutic oncolytic viruses, including but not limited to, retroviruses, picornaviruses, rhabdoviruses, paramyxoviruses, reoviruses, parvoviruses, adenoviruses, herpesviruses, and poxviruses.

In another embodiment, the CAR-DCs or a population of CAR-DCs of the present disclosure may be used in combination with immunostimulatory therapies, such as toll-like receptors agonists, including but not limited to, TLR3, TLR4, TLR7 and TLR9 agonists.

In another embodiment, the CAR-DCs or a population of CAR-DCs of the present disclosure may be used in combination with stimulator of interferon gene (STING) agonists, such as cyclic GMP-AMP synthase (cGAS).

### III. Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to DC cells, DC progenitors, DC precursors, or modified cells thereof, CAR constructs, or CAR-DC cells or a nucleic acid sequence encoding a CAR construct, and delivery systems. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or from a randomized group of subjects. A reference value can be used in place of a control or reference sample, which was previously obtained from a healthy subject or a group of healthy subject. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer implemented method or methods for performing such computer-implemented method or methods, and can also be embodied in the form of a tangible or non-transitory computer readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D.N. Glover ed. 1985); Nucleic Acid Hybridization (B.D. Hames & S.J. Higgins eds.(1985»; Transcription and Translation (B.D. Hames & S.J. Higgins, eds. (1984»; Animal Cell Culture (R.I. Freshney, ed. (1986»; Immobilized Cells and Enzymes (IRL Press, (1986»; and B. Perbal, A practical Guide To Molecular Cloning (1984); F.M. Ausubel et al. (eds.).

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ± 5%, but can also be ± 4%, 3%, 2%,1%, etc. Whether or not modified by the term “about,” the claims include equivalents to the quantities.

When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements.

The terms “patient,” “subject,” “individual,” and the like are used interchangeably herein, and refer to any animal or cells thereof whether in vitro or in situ, amenable to the methods described herein. In certain non-limiting embodiments, the patient, subject or individual is a human.

As used herein, the term “subject” refers to a mammal, preferably a human. The mammals include, but are not limited to, humans, primates, livestock, rodents, and pets. A subject may be waiting for medical care or treatment, may be under medical care or treatment, or may have received medical care or treatment.

Described herein is a method of generating chimeric antigen receptor dendritic cells (CAR-DCs).

Precursor cells, for example, stem cells, monocytes, or in this case bone marrow cells were isolated, grown in Flt3L for about one day, then virally transduced with a CAR of interest, then further differentiated with Flt3L for about 2-15 days to generate DC-like cells for use in vivo or in vitro. CAR expression can be assessed using an antibody that recognizes the CAR on the cell surface by FACS analysis. Viral transduction was done here using retrovirus or lentivirus, but may be achieved with any gene delivery method.

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site, all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A constructs of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 50-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity = X/Y100, where X is the number of residues scored as identical matches by the sequence alignment program’s or algorithm’s alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6 X SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65° C. in the salt conditions of a 6 X SSC, then the two sequences will not hybridize. On the other hand, if the melting temperature is above 65 □C in the same salt conditions, then the sequences will hybridize. In general, the melting temperature for any hybridized DNA:DNA sequence can be determined using the following formula: Tm = 81.5° C. + 16.6(log10[Na+]) + 0.41 (fraction G/C content) -0.63(% formamide) - (600/I). Furthermore, the Tm of a DNA:DNA hybrid is decreased by 1-1.5□C for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| | | |
|---|---|---|
| Side Chain Characteristic | | Amino Acid |
| Aliphatic Non-polar | G A P I L V | |
| Polar-uncharged | C S T M N Q | |
| Polar-charged | D E K R | |
| Aromatic | H F W Y | |
| Other | N Q D E | |

Conservative Substitutions II

| | |
|---|---|
| Side Chain Characteristic Non-polar (hydrophobic) | Amino Acid |
| A. Aliphatic: | A L I V P |
| B. Aromatic: | F W |
| C. Sulfur-containing: | M |
| D. Borderline: | G |

Uncharged-polar

| | |
|---|---|
| A. Hydroxyl: | S T Y |
| B. Amides: | N Q |
| C. Sulfhydryl: C | |
| D. Borderline: | G |
| Positively Charged (Basic): | K R H |
| Negatively Charged (Acidic): | D E |

Conservative Substitutions III

| | | |
|---|---|---|
| Original Residue | | Exemplary Substitution |
| Ala (A) | Val, Leu, Ile | |
| Arg (R) | Lys, Gln, Asn | |
| Asn (N) | Gln, His, Lys, Arg | |
| Asp (D) | Glu | |
| Cys (C) | Ser | |
| Gln (Q) | Asn | |
| Glu (E) | Asp | |
| His (H) | Asn, Gln, Lys, Arg | |
| Ile (I) | Leu, Val, Met, Ala, Phe, | |
| Leu (L) | Ile, Val, Met, Ala, Phe | |
| Lys (K) | Arg, Gln, Asn | |
| Met(M) | Leu, Phe, Ile | |
| Phe (F) | Leu, Val, Ile, Ala | |
| Pro (P) | Gly | |
| Ser (S) | Thr | |
| Thr (T) | Ser | |
| Trp(W) | Tyr, Phe | |
| Tyr (Y) | Trp, Phe, Tur, Ser | |
| Val (V) | Ile, Leu, Met, Phe, Ala | |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326 - 330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

The term “activation” (and other conjugations thereof) in reference to cells is generally understood to be synonymous with “stimulating” and as used herein refers to an enhanced functional outcome and/or expansion of cell populations.

The term “antigen” as used herein in the context of a CAR target is a cell surface protein recognized by (i.e., that is the target of) chimeric antigen receptor. In the classical sense antigens are substances, typically proteins, that are recognized by antibodies or the T-cell receptor, but the definitions overlap insofar as the CAR comprises antibody-derived domains such as light (VL) and heavy (VH) chains recognizing one or more antigen(s). An antigen can also comprise any intracellular or surface molecule, generally a protein or peptide, capable of being recognized by the immune system (most frequently T-cells, or antibodies).

The term “cancer” refers to a malignancy or abnormal growth of cells in the body. Many different cancers can be characterized or identified by particular cell surface proteins or molecules. Thus, in general terms, cancer in accordance with the present disclosure may refer to any malignancy that may be treated with an immune effector cell, such as a CAR-DCs as described herein, in which the modified dendritic cell recognizes and binds to the cell surface protein on the cancer cell. As used herein, cancer may refer to a hematologic malignancy, such as multiple myeloma, a T-cell malignancy, or a B cell malignancy. T cell malignancies may include, but are not limited to, T-cell acute lymphoblastic leukemia (T-ALL) or non-Hodgkin’s lymphoma. A cancer may also refer to a solid tumor, such as including, but not limited to, cervical cancer, pancreatic cancer, ovarian cancer, mesothelioma, and lung cancer.

A “cell surface protein” as used herein is a protein (or protein complex) expressed by a cell at least in part on the surface of the cell. Examples of cell surface proteins include the TCR (and subunits thereof) and CD7.

A “chimeric antigen receptor” or “CAR” as used herein and generally used in the art, refers to a recombinant fusion protein that has an extracellular ligand-binding domain, a transmembrane domain, and a signaling transducing domain that directs the cell to perform a specialized function upon binding of the extracellular ligand-binding domain to a component present on the target cell. For example, a CAR can have an antibody-based specificity for a desired antigen (e.g., tumor antigen) with a T cell receptor-activating intracellular domain to generate a chimeric protein that exhibits specific anti-target cellular immune activity. First-generation CARs include an extracellular ligand-binding domain and signaling transducing domain, commonly CD3ζ or FcεRIγ. Second generation CARs are built upon first generation CAR constructs by including an intracellular costimulatory domain, commonly 4-1BB or CD28. These costimulatory domains help enhance CAR-T cell cytotoxicity and proliferation compared to first generation CARs. The third generation CARs include multiple costimulatory domains, primarily to increase CAR-T cell proliferation and persistence. Chimeric antigen receptors are distinguished from other antigen binding agents by their ability both to bind MHC-independent antigens and transduce activation signals via their intracellular domain.

The term “composition” as used herein refers to an immunotherapeutic cell population combination with one or more therapeutically acceptable carriers.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

As various changes could be made in the above-described materials and methods without departing from the scope of the invention, it is intended that all matter contained in the above description and in the examples given below, shall be interpreted as illustrative and not in a limiting sense.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Generation and Characterization of Functional Car Dendritic Cells

Dendritic cells (DCs) are critical in initiating an adaptive immune response. Numerous studies demonstrate that DCs, are limited in the tumor microenvironment, and even in cancer patients in general (Hegde S, et al. Cancer Cell 2020;37:289-307 e9). Further, even if DCs are present, they can induce either tolerance or rejection of an antigen, and they generally have no strong signal that tells them a tumor cell is “bad” and should be eliminated.

Currently we are in the age of cellular therapy, whereby a patient’s own cells can be collected, modified to exert a specific function, amplified, and injected back into the patient to treat his or her disease. This presents a new opportunity for DC activation, and reliably eliciting an adaptive immune response. The current standard treatment have focused on systemically injecting a molecule to activate DCs throughout the body. Contrary to systemically activating DCs, he present example examines if DCs can be collected, genetically modified with a CAR to recognize a cancer cell, and that recognition can induce signaling pathways that instruct the cell to engulf the target, present its antigens, and secrete additional immune activating molecules of interest, depending on how the CAR is constructed.

Prior work has been done to create CAR-macrophages or CAR-phagocytes. Macrophages, like dendritic cells, can phagocytose material, and can present antigens. However, in vivo, macrophages are unable to effectively cross present tumor cell-associated antigens, and are unable to create a tumor-eliminating immune response. In vivo, DCs, and particularly the subset of DCs known as type 1 conventional dendritic cells (cDC1s), are the only cells capable of effective tumor antigen cross-presentation, as evidenced by the fact that in the absence of cDC1s an adaptive antitumor response is not achievable, an anti-tumor immune response cannot be mounted, and tumor cannot be eliminated by the immune system in vivo (Theisen DJ, et al. Science 2018;362:694-9; and Hildner K, et al. Science 2008;322:1097-100). Thus, conceptually, CAR-macrophages could potentially achieve the goal of direct tumor phagocytosis or possibly direct cellular cytotoxicity against tumors with homogeneous antigen expression. However, they would not be expected to achieve the goal of antigen cross presentation or an adaptive anti-tumor T cell response, consistent with published data (Morrissey MA, et al. Elife 2018;7).

CAR-macrophages have been created by fusing the intracellular domain of various macrophage receptors that induce phagocytosis, such as Fc Receptors, toll-like receptors, or other macrophage or T-cell based receptors, with a tumor-recognizing scFv extracellular domain. No CARs to date have successfully been created that endow cells with cDC1 capacity; that is, the ability to cross-prime an anti-tumor T-cell response of endogenous T cell populations.

The present Example provides an intracellular signaling domain that generates functional CAR-DCs, which, unlike previously described CARs, endows the transduced myeloid cell with the capacity to cross-present phagocytosed tumor antigen in a manner that cross-primes endogenous T-cells to mount a strong and successful adaptive anti-tumor response, in vitro and in vitro.

The immediate implication of these studies provides a new therapeutic strategy to directly eliminate antigen positive (Ag⁺) tumors targeted by the CARs, and indirectly eliminate CAR-Ag⁻ tumor cells (not recognized by the CAR), through cross-presentation and epitope spreading.

The present example describes a method of making chimeric antigen receptor dendritic cells (CAR-DCs) and their resulting functionality. CAR constructs were cloned for their ability to differentiate DCs and for their antigen cross-presentation functionality. Evidence was obtained that one particular CAR construct was able to successfully drive tumor engulfment and cross presentation of endogenous tumor antigen to stimulate anti-tumor CD8 T cells. This FMS-like tyrosine kinase 3 (Flt3)-based CAR is described below.

### Methods

Various CAR constructs were created with various intracellular signaling domains, introduced into DC precursors, and were screened for their ability to sustain the phenotype of a cross-presenting DC after transduction, and to functionally cross-present tumor antigen. These data include a direct comparison of CARs that are Fc receptor-based, toll-like receptor (TLR)-based, or Flt3-based. One particular construct has emerged that most successfully endows the cell with a cDC1 phenotype that maintains tumor-specific uptake capacity and possesses the ability to cross present tumor antigen: the Flt3-based CAR.

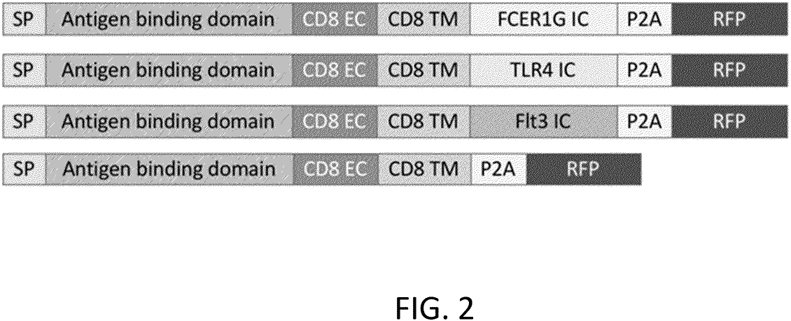

The design of this CAR is as follows: a signal peptide that drives surface expression, followed by a tumor binding domain (generally an scFv from an antibody), followed by an extracellular domain, transmembrane domain, and intracellular domain (see e.g., FIG. 2). The domain critical for achieving successful CAR-DCs is an intracellular domain derived from Flt3.

Tumor models: Unless otherwise stated, all experiments are performed in the MCA-induced soft tissue flank sarcoma model, which was demonstrated to contain tumor antigens that are recognized by T-cells (Gubin MM, et al. Nature 2014;515:577-81; and Alspach E, et al. Nature 2019;574:696-701), which are primed and cross-primed by DCs (Ferris ST et al. Nature 2020;584:624-9). As a second model the C57BL/6 KPC (Kras(G12D/+);p53(R172H/+);Pdx-1-Cre) pancreatic cancer model was used because like the human cancer it has low mutational burden and is poorly immunogenic, it creates a sophisticated immunosuppressive tumor microenvironment (Tseng WW, et al. Clin Cancer Res 2010;16:3684-95), and can be injected orthotopically or subcutaneously. The pancreatic tumor antigen EphA2 is expressed on 90% of pancreatic cancers and more rarely on normal tissues. Mouse KPC tumor cells as well as MCA-induced sarcoma cells naturally express EphA2. zsGreen and ovalbumin (ova) were introduced into these cells in order to quantify the T-cell response using T-cells derived from OT1 mice, which are transgenic mice that generate cytotoxic CD8 T-cells that specifically recognize ova peptide presented on MHC-I. For in vivo models, tumor cells were injected into the bilateral flank, which is the natural site of disease for the MCA-induced sarcoma. After three days of tumor establishment, mice were treated with local injection of CAR-transduced cells. Mice were sacrificed when tumors reached 2 cm diameter.

DC and CAR-DC generation. Bone marrow cells were isolated by flushing the bone marrow of syngeneic mice, and grown in Flt3L for one day, then transduced with the CAR of interest or empty viral vector, then further differentiated with Flt3L (80 ng/ml) for 6-10 days to generate differentiated DCs. cDC1 and cDC2 populations were quantified by FACS using a standard gating strategy in which cDCs are lineage-negative, B220⁻, CD11c⁺, MHC-II⁺, and cDC1 and cDC2s are further differentiated by CD24 and Sirpa positivity, respectively. CAR expression was assessed using an anti-human Fab2 antibody that recognizes the CAR on the cell surface by FACS analysis.

Macrophage and CAR-macrophage generation. Bone marrow cells were isolated as above, grown in M-CSF or GM-CSF for one day, then transduced with the CAR of interest, then further differentiated with M-CSF or GM-CSF for 5-10 days to generate virally transduced macrophages, as previously described. CAR expression was assessed using an antibody that recognizes the CAR on the cell surface by FACS analysis.

T cell activation and proliferation. CD3, CD4 and CD8 T cell markers were assessed by FACS analysis for proliferation using CFSE as previously described (Theisen DJ, et al. Science 2018;362:694-9).

T cell function. T cell function was assessed by the cytotoxic T cell lymphocyte assay (CTL), where a defined ratio of effector T cells is mixed with target tumor cells, and in some cases antigen presenting cells, for the indicated amount of time. In multi-day experiments, 50% of the media was changed every 2 days. Remaining tumor cell number was quantified using BioTek Cytation 5 live cell imaging and image analysis software.

CAR-induced tumor phagocytosis. Phagocytosis was assessed by genetically labeling the target cells with an acid-resistant fluorophore (zsGreen), labeling the CAR-transduced cells with RFP (which is delivered by the same vector that delivers the CAR), then coculturing the cells and quantifying phagocytosis by FACS or by direct live video microscopy using BioTek’s Cytation 5 imaging and software (both of which quantify, in different ways, the number of red cells that phagocytose or take up green cells).

Cross presentation assays. Standard cross presentation assays were performed by mixing CAR-transduced or control DCs with ova-expressing tumor or ova-expressing heat killed listeria bacteria, then adding CFSE-labeled OT1 T cells (which react against ova SIINFEKL peptide presented on MHC-I), and measuring CD8 T cell proliferation by flow cytometry after 3 days.

### Results

### (I) CAR Macrophages Fail to Induce a Systemic Immune Response

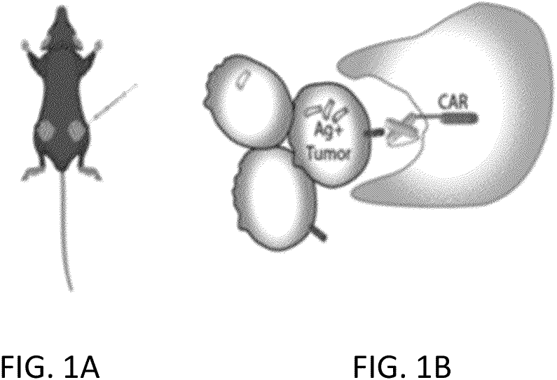

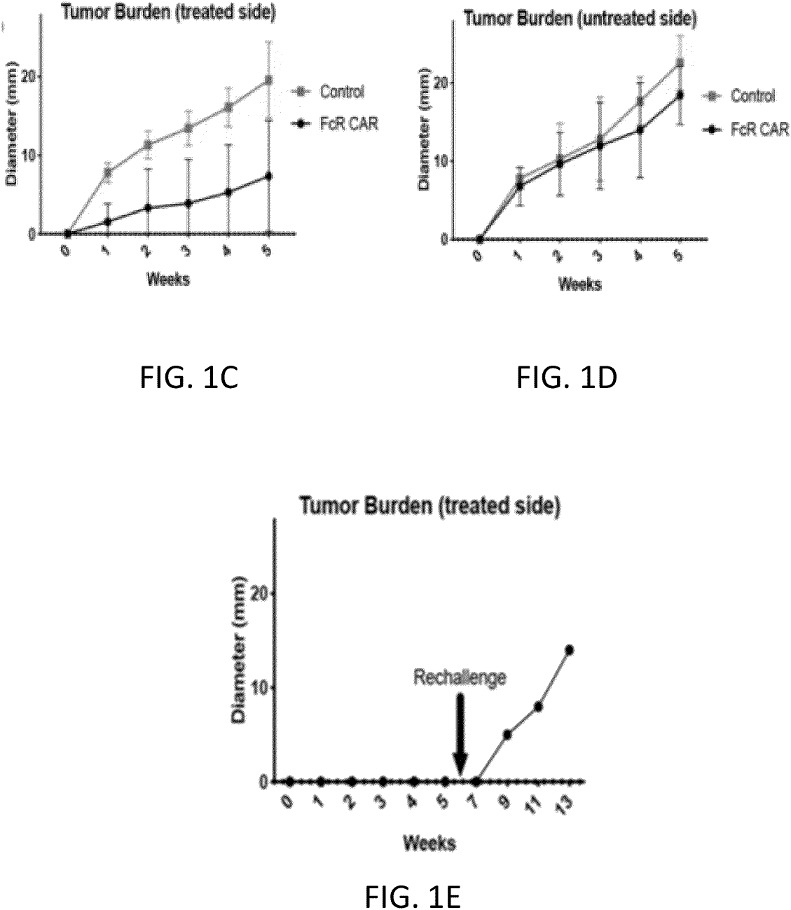

Since macrophages, including CAR macropahges, have the ability to stimulate T-cells in vitro (Klichinsky M, et al. Nat Biotechnol 2020;38:947-53), and since macrophages are professional antigen presenting cells, the hypothesis that CAR-macrophages can induce a systemic immune response was tested. In this model, tumor cells were injected into the bilateral flank of syngeneic mice, and allowed to establish for three days. After, three days CAR macrophages were injected into one tumor (FIG. 1A). If the CAR macrophages locally phagocytose and/or kill tumor, the locally injected tumor is expected to respond. If they take up antigen and cross-prime T-cells effectively, the T-cells would circulate and if effectively stimulated would be expected to eliminate the contralateral tumor as well as the local tumor. If tumor response is not observed on either side, neither is occurring. A FcR CAR was utilized, which has previously been found to effectively induce phagocytosis by engineered macrophages (conceptually depicted in FIG. 1B)(Morrissey MA et al. Elife 2018;7; and Klichinsky M et al. Nat Biotechnol 2020;38:947-53). As was used in previous studies the control treatment in these experiments was untransduced macrophages. It was demonstrated that FcR CAR macrophages significantly reduce tumor volume in the site of injection (FIG. 1C), but fail to induce any degree of distal tumor elimination (FIG. 1D), consistent with local killing effect without inducing an adaptive immune response. One FcR CAR macrophage-treated mouse exhibited complete tumor elimination at the site of injection. In this mouse, if an adaptive immune response contributed to rejection, it would be expected that re-challenge with the same tumor would result in absence of tumor growth. Upon re-injecting tumor in this mouse, tumor grew out, consistent with lack of an adaptive immune response generated by the CAR macrophage (FIG. 1E).

### (Ii) Generation of CAR DCs

Given this result, Applicants endeavored to generate CAR modified cells that could induce an effective systemic anti-tumor immune response upon local tumor encounter. CARs are designed in a standard manner in which the extracellular domain recognizes a tumor antigen, the extracellular hinge and transmembrane domains are constant (CD8, in these experiments) while the internal signaling domain varies. It was hypothesized that different internal domains may assist in promoting cross-priming of T-cells to internalized tumor antigen. Since bacteria are a common pathogen recognized by antigen presenting cells with a specific receptor, TLR4, that recognizes LPS, it was hypothesized that a CAR with a TLR4 signaling domain may improve the ability of CAR modified myeloid cells to cross-prime T-cells. Given the potency of cDCs in T-cell cross-priming, it was also hypothesized that a CAR that induced Flt3 signaling, which is the receptor most critical to initiating and maintaining cDC differentiation, may improve the cross-priming capacity of engineered myeloid cells. In these direct comparison experiments, the internal domain thus consists of an Fc Receptor signaling domain (here, the common gamma chain), a toll like receptor (TLR) signaling domain (TLR4 in this case), or a Flt3 signaling domain (FIG. 2). As a control, a CAR was created that has identical extracellular and transmembrane domains, but no internal domain, so it binds to tumor but does not signal. Each CAR also expresses RFP following a P2A sequence to assess transduction efficiency.

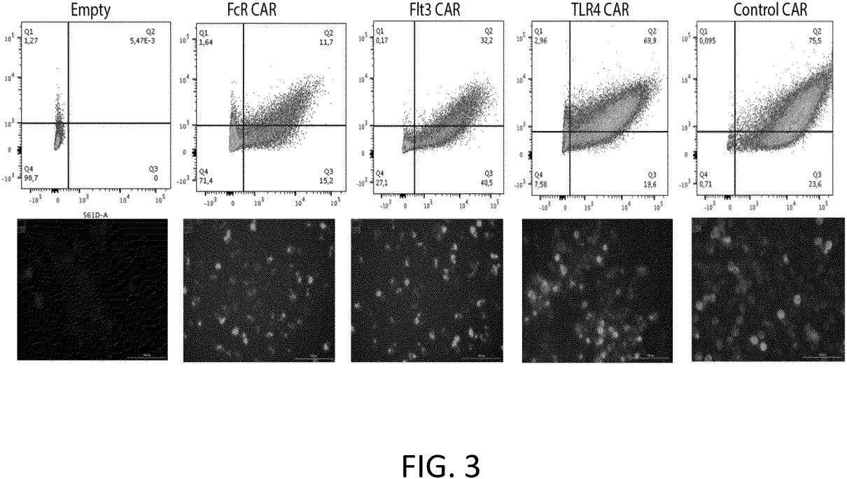

After substantial viral production and transduction optimization, successful CAR transduction was achieved and surface expression, confirmed by flow cytometry using an antibody that directly binds the CAR’s scFv, as well as internal fluorescence from the introduced RFP (FIG. 3).

### (Iii) Flt3 Based CAR Induces Cell Proliferation After Tumor-CAR Coculture

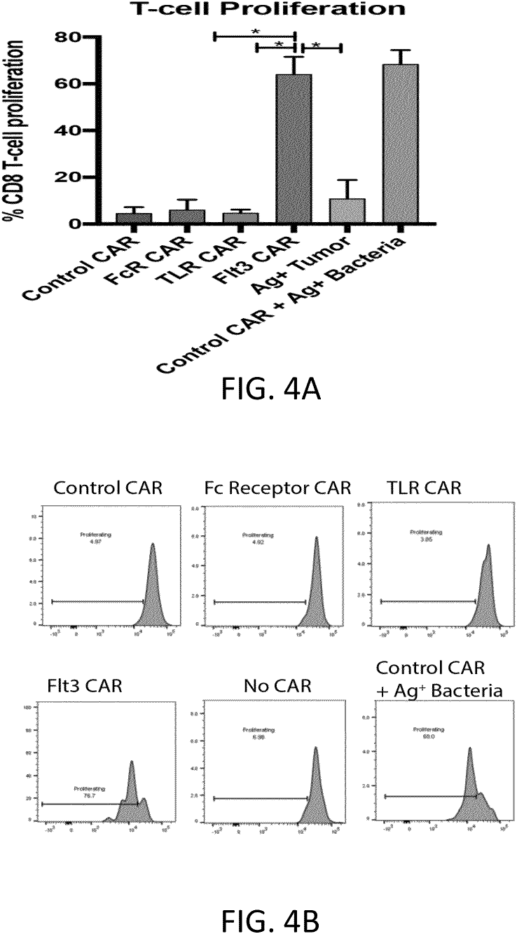

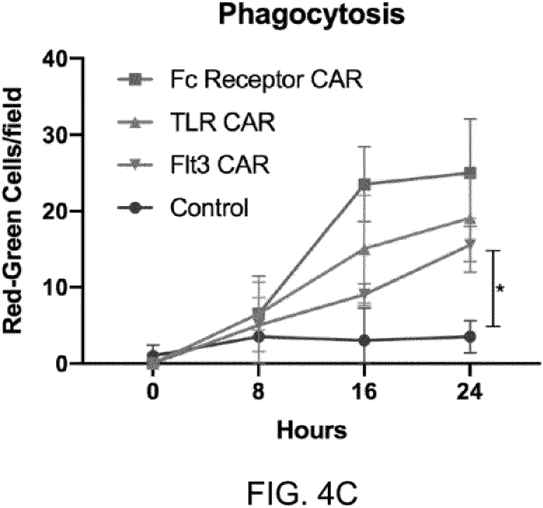

Conceptually, tumor phagocytosed by CAR-transduced cells may be degraded, or processed into peptides and cross-presented to cross-prime CD8 T-cells. Using a cross-presentation assay in which OT1 T-cells are mixed with tumor cells and the antigen presenting cells, it was first found that DCs containing a control CAR (containing an extracellular and transmembrane domain, but no intracellular signaling domain), mixed with ova-expressing heat killed listeria bacteria and OT1 T-cells produce a strong T cell proliferative response, as expected (positive control) (FIG. 4A). However, when ova-expressing bacteria are replaced with ova-expressing tumor cells, control CAR-transduced cells produce no measurable T cell response. Surprisingly, despite the fact that Fc Receptor and TLR based CARs provide signaling domains that potentially recapitulate bacterial or opsonized pathogen-induced signaling, these CARs fail to induce more tumor-antigen-specific T-cell proliferation than the control CAR. On the other hand, the Flt3 based CAR successfully induces tumor-antigen-specific T-cellproliferation after tumor-CAR coculture, similar to the level induced by antigen-expressing bacteria (FIG. 4). These data demonstrate that the Flt3 CAR facilitates a tumor antigen-specific T-cell response similar in magnitude to the robust response a DC generates against bacteria, while conventional phagocytic CARs generate no greater tumor antigen-specific T-cell response than a control non-signaling CAR after encountering tumor, despite achieving significant tumor phagocytosis (FIG. 4C).

### (Iv) Flt3 CAR DCs Achieve Significantly More Tumor Eradication Compared with Fc Receptor or TLR Based CARs

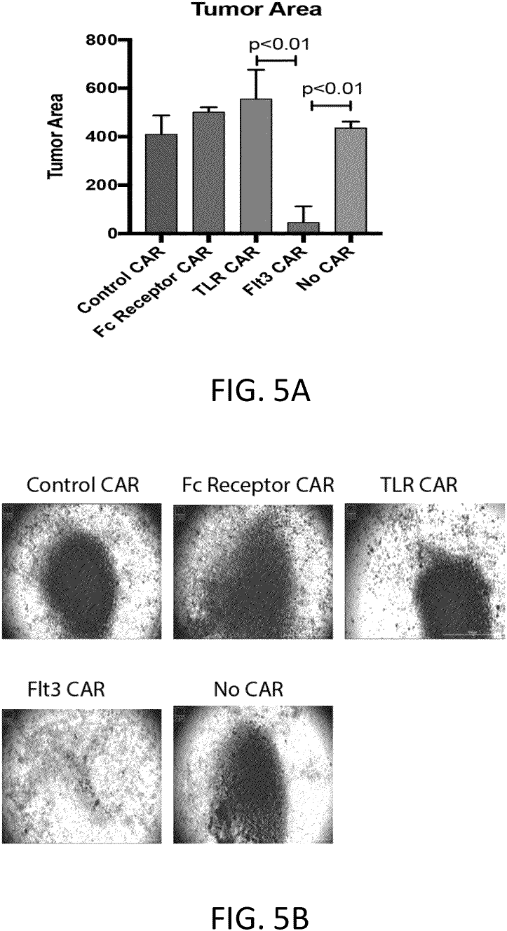

To test whether the antigen cross presentation achieved by the CAR antigen presenting cells is functionally meaningful, zsGreen+ Ova antigen-expressing tumor cells were incubated with the indicated CAR transduced DC-differentiated cells in addition to OT1 T-cells in a 2:1:1 ratio. Tumor cell area was quantified 10 days later by BioTek live cell imaging and imaging software that quantifies GFP tumor area. Consistent with increased T-cell activation by the Flt3 CAR DC, Flt3 CAR DCs achieve significantly more tumor eradication compared with Fc Receptor or TLR based CARs (FIG. 5).

### (V) Flt3 CAR Improves DC Cell Survival and Differentiation

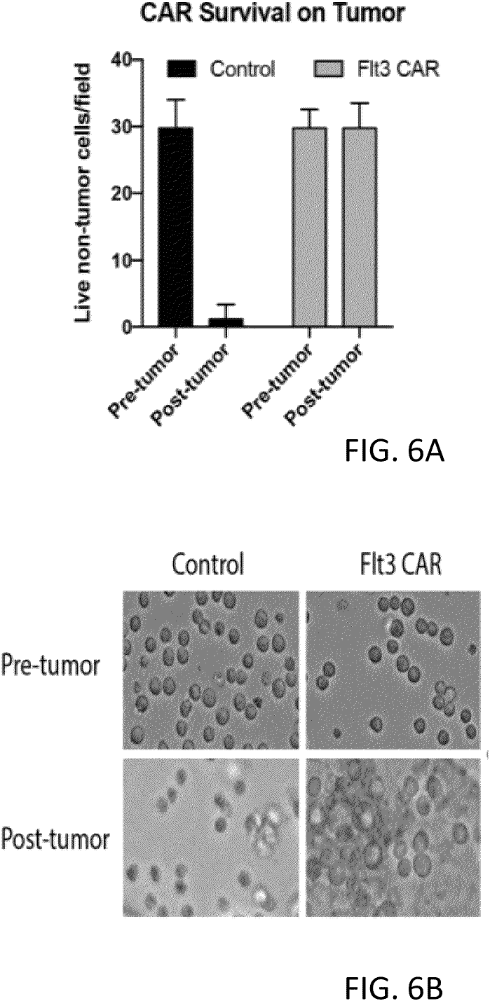

To ascertain why Flt3 CAR DCs are more effective at antigen cross presentation, it was hypothesized that this particular CAR may either improve the ability of the cell to survive, or to differentiate into the correct cell phenotype for effective T-cell cross-priming, upon tumor encounter and CAR signaling. It was observed, in the presence of tumor, that Flt3 CAR expressing cells appeared to have a survival advantage. To definitively test whether CAR-induced Flt3 signaling provided significant enough signaling to maintain cell survival on its own, a HoxB8 DC cell line was obtained that critically depends on Flt3 ligand for survival; without Flt3 ligand, these cells do not survive in culture, but with Flt3 ligand they survive and can differentiate into DCs that are equivalent to wild type DCs. These cells were transduced with a control CAR that provides no signaling as well as Flt3 CAR, initially in the presence of exogenous Flt3 ligand, then these cells were plated on tumor cells in normal growth media without Flt3 ligand. After two days, all of the control CAR HoxB8 cells had died and were uniformly distributed across the well; however, Flt3 CAR HoxB8 cells were clumped around tumor cells and continued to survive healthily (FIG. 6). This demonstrates that the Flt3 CAR provides significant survival signaling through its Flt3 CAR signaling domain. In the tumor microenvironment, where little Flt3 ligand is present and DC survival is poor, this tumor-induced CAR survival signaling may provide further advantage to the CAR DCs.

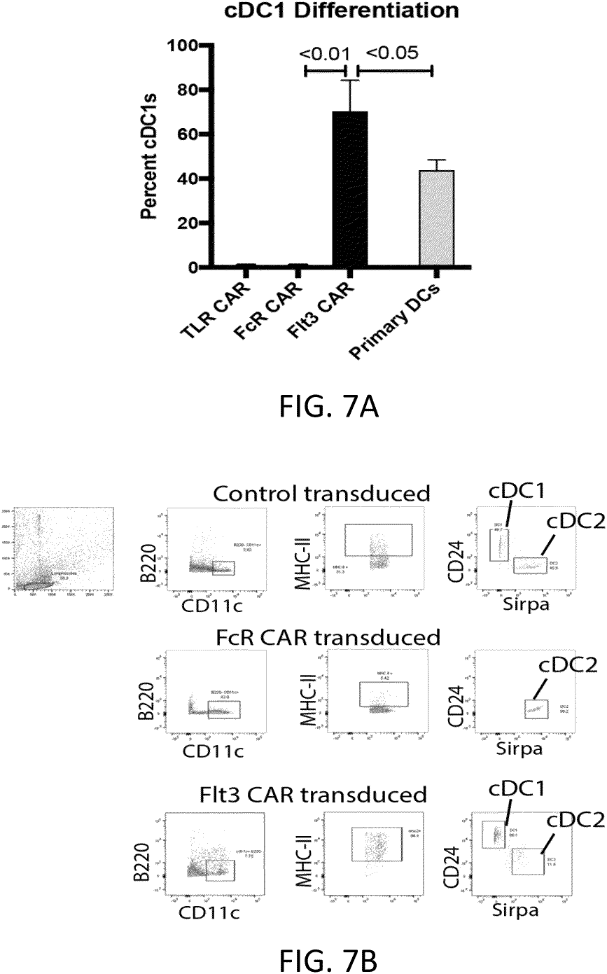

It was next tested whether Flt3 CAR transduced cells differentiated differently compared with identical cells transduced with the TLR4 or FcR CARs. After differentiation in Flt3 ligand, cells were phenotyped by flow cytometry. Since cDC1 cells are the critical DC for generating an adaptive immune response and for tumor elimination, the cDC1 phenotype was specifically examined. Surprisingly, the presence of TLR4- or FcR-based signaling in the CAR substantially reduced the ability of DC precursors to successfully differentiate into cDC1s, even in the presence of the Flt3 ligand (FIG. 7). The presence of these inflammatory signaling CARs appears to divert the cells to a macrophage or cDC2 phenotype, even in the presence of cDC1-differentiating growth factors. However, the presence of the Flt3 CAR maintained, if not enhanced, the ability of cDC1s to successfully differentiate (FIG. 7).

These data show Flt3 based CARs are uniquely able to produce true, functional cDC1s, which has not been before demonstrated. These are thus true “CAR-DCs,” which are to be distinguished from CAR-macrophages or CAR-phagocytes that possess significantly inferior ability to cross-prime T-cells.

### (Vi) FLT3 CAR DCs Generate a Robust Adaptive Immune Response

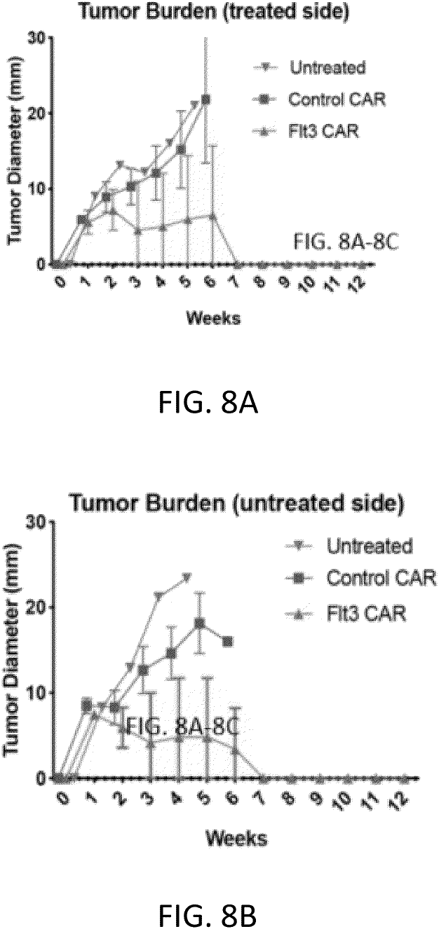

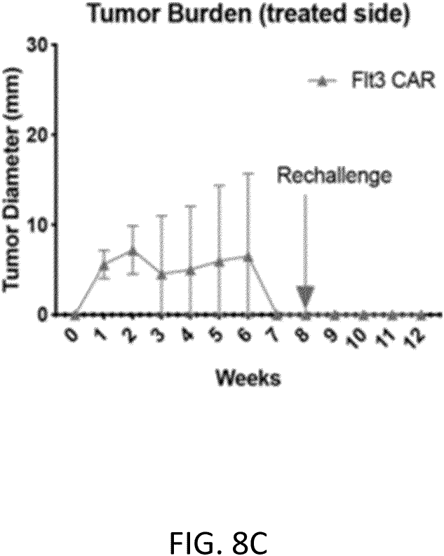

To test the capacity of Flt3 CAR DCs to generate a robust adaptive immune response that eliminates distant tumor in vivo, the dual flank orthotopic sarcoma tumor model was employed. Tumor was injected into the bilateral flank and allowed to establish in syngeneic mice for three days. Control non-signaling CAR DCs or Flt3 CAR DCs were then injected into one of the two tumor sites, and tumor growth at both sites was quantified over time. It was found that mice treated with control CAR DCs progressed at both the site of CAR DC injection and the untreated site (FIG. 8A). Flt3 CAR treated tumor continued to grow for over a week after CAR DC local injection, then began to regress. With similar kinetics, consistent with an adaptive immune response, the distal tumor sites also grew for 1-2 weeks after Flt3 CAR DC treatment, then similarly began to regress. By week seven, tumor was undetectable in all local and distal tumor sites of Flt3 CAR DC treated mice (FIG. 8B), while control CAR and untreated mice had all progressed to the point of death by the same time point. To test whether Flt3 CAR DC-treated mice had achieved a successful adaptive immune response with immunologic memory, tumor was re-injected into the flank of these mice. All Flt3 CAR DC-treated mice were protected from tumor rechallenge, as tumor was unable to regrow in any of these mice (FIG. 8C). These data demonstrate the Flt3 CAR DCs successfully elicit an adaptive systemic immune response against the targeted tumor, which is robust enough to eliminate distant established tumor. The Flt3 CAR DC-induced anti-tumor response persists and continues to provide immunity to tumor rechallenge.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All references, patents and patent applications disclosed herein are incorporated by reference with respect to the subject matter for which each is cited, which in some cases may encompass the entirety of the document.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

What is claimed is:

1. A modified cell comprising a chimeric antigen receptor (CAR), wherein the CAR comprises:
an antigen binding domain;
a transmembrane domain;
an intracellular domain comprising a FMS-like tyrosine kinase 3 (Flt3) signaling domain; and
the modified cell is a dendritic cell or a precursor or a progenitor cell thereof.

2. A chimeric antigen receptor (CAR) construct comprising:
(i) an antigen-binding domain;
(ii) a transmembrane domain; and
(iii) an intracellular signaling domain comprising an FMS-like tyrosine kinase 3 (Flt3) signaling domain,
wherein the CAR construct is capable of being expressed or functioning in a dendritic cell (DC) or a precursor or a progenitor cell thereof.

3. A modified dendritic cell comprising the CAR construct of claim 2, wherein the dendritic cell is selected from a cDC1 cell or precursor or progenitor thereof.

4. A modified cell comprising a first nucleic acid sequence encoding the CAR of claim 2 or a second nucleic acid sequence encoding the antigen binding domain, the transmembrane domain, and the intracellular domain.

5. The modified cell of claim 4, wherein the first intracellular nucleic acid sequence encodes a protein product comprising Flt3 or a Flt3-based protein product or subsequent intracellular nucleic acid sequence encodes a protein product comprising Flt3 or a Flt3-based protein product.

6. The modified cell of claim 1, wherein the CAR further comprises a signal peptide or an additional extracellular domain.

7. The modified cell of claim 1, wherein the modified cell is a conventional type 1 dendritic cell (cDC1).

8. The modified cell of any one of claims 1 or 3, wherein the progenitor cell is selected from a peripheral blood mononuclear cell (PBMC), a monocyte and dendritic cell progenitor (MDP), a common myeloid progenitor (CMP), a lymphoid-primed multipotent progenitor (LMPP) or a common dendritic cell progenitor (CDP), or a stem cell.

9. The modified cell of claim 1, wherein the modified cell is capable of antigen cross-presentation, an adaptive antitumor immune response, or activation of antitumor T cells.

10. The modified cell of claim 1, wherein the antigen binding domain comprises an antibody or fragment thereof.

11. The modified cell of claim 10, wherein the antibody has a binding affinity to a tumor cell antigen.

12. The modified cell of claim 11, wherein the tumor cell antigen is EphA2.

13. The modified cell of claim 1 or 3, wherein the antigen binding domain is against a disease-associated antigen, selected from EphA2, EGFRviii, AFP, CEA, CA-125, MUC-1, CD123, CD30, SlamF7, CD33, EGFRvIII, BCMA, GD2, CD38, PSMA, B7H3, EPCAM, IL-13Ra2, PSCA, Mesothelin, Her2, CD19, CD20, CD22, sial-LewisA, LewisY, CIAX, or another tumor-enriched protein.

14. The modified cell of any one of claims 1 or 3, wherein the modified cell is capable of selectively engulfing tumor cells, cross-presenting a tumor antigen, and activating T-cells to respond to the tumor antigen.

15. The modified cell of any one of claims 1 or 3, wherein the modified cell is capable of cross-presenting tumor antigens (or having tumor antigen cross-presentation), wherein antigen cross-presentation is the ability of a cell to present internalized antigens on type I major histocompatibility complex molecules (MHC I), which is necessary for an efficient adaptive immune response against tumor cells.

16. The modified cell of any one of claims 1 or 3, wherein the modified cell is capable of eliminating antigen positive (Ag⁺) tumors targeted by the CARs, and indirectly eliminate CAR-Ag⁻ solid tumor cells (not recognized by the CAR), through epitope spreading.

17. A pharmaceutical composition comprising the modified cell of claim 1.

18. A method of stimulating an adaptive antitumor T cell response in a subject comprising:
administering to the subject a therapeutically effective amount of a pharmaceutical composition comprising a chimeric antigen receptor dendritic cell (CAR-DC); wherein,
the CAR comprises an antigen binding domain, a transmembrane domain, and an intracellular domain;
the intracellular domain comprises a FMS-like tyrosine kinase 3 (Flt3) signaling domain; and
the cell is a dendritic cell or a progenitor cell thereof.

19. The method of claim 18, wherein the subject has a proliferative disease, disorder, or condition (e.g., cancer).

20. The method of claim 18, wherein the method induces phagocytosis of cancer cells in a subject.

21. The method of claim 18, wherein the CAR-DC cross-primes an anti-tumor T-cell response.

22. The method of claim 18, wherein the CAR-DC creates a tumor-eliminating immune response.

23. The method of claim 19, wherein the proliferative disease, disorder, or condition is a malignant tumor, a solid tumor, or liquid tumor.

24. The method of claim 19, wherein the modified cell
directly targets antigen positive (Ag⁺) tumor cells for elimination; or
indirectly targets CAR-antigen negative (Ag⁻) tumor cells for elimination through cross-presentation and epitope spreading.

25. A method of making a population of modified immune cells (e.g., DCs, cDC1s), comprising:
(i) providing or having been provided a population of cells from a subject (e.g., mononuclear or stem cells from circulation, cord, or bone marrow);
(ii) culturing the population of cells in a medium comprising an FMS-like tyrosine kinase 3 (Flt3) agonist for at least about one day;
(iii) introducing a Flt3-based chimeric antigen receptor (CAR) into the cells from (ii); and
(iv) culturing the cells from (iii) in a medium comprising an FMS-like tyrosine kinase 3 (Flt3) agonist for an amount of time sufficient to form a modified cell, wherein,
the CAR comprises an antigen binding domain, a transmembrane domain, and an intracellular domain, the intracellular domain comprising a FMS-like tyrosine kinase 3 (Flt3) singling domain.

26. The method of claim 25, wherein the amount of time sufficient to form the modified cell is between about 5 days and about 15 days.

27. The method of claim 25, wherein introducing the CAR into the bone marrow cells comprises introducing an intracellular nucleic acid sequence encoding a protein product comprising an Flt3 or an Flt3-like intracellular domain into the cells.

28. The method of claim 27, wherein the modified cell is capable of antigen cross-presentation, an adaptive antitumor immune response, or activation of antitumor T cells.

29. The method of claim 25, wherein the modified cell is a dendritic cell or a conventional type 1 dendritic cell (cDC1).


SEQUENCE LISTING (2 sequences)

SEQ ID NO: 1 - protein, 430 aa, Artificial Sequence
  Synthesized
HKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

SEQ ID NO: 2 - protein, 428 aa, Artificial Sequence
  Synthesized
HKYKKQFRYESQLQMIQVTGPLDNEYFYVDFRDYEYDLKWEFPRENLEFGKVLGSGAFGRVMNATAYGISKTGVSIQVAVKMLKEKADSCEKEALMSELKMMTHLGHHDNIVNLLGACTLSGPVYLIFEYCCYGDLLNYLRSKREKFHRTWTEIFKEHNFSFYPTFQAHSNSSMPGSREVQLHPPLDQLSGFNGNSIHSEDEIEYENQKRLAEEEEEDLNVLTFEDLLCFAYQVAKGMEFLEFKSCVHRDLAARNVLVTHGKVVKICDFGLARDILSDSSYVVRGNARLPVKWMAPESLFEGIYTIKSDVWSYGILLWEIFSLGVNPYPGIPVDANFYKLIQSGFKMEQPFYATEGIYFVMQSCWAFDSRKRPSFPNLTSFLGCQLAEAEEAMYQNMGGNVPEHPSIYQNRRPLSREAGSEPPSPQAQ